(19) United States
(12) Patent Application Publication
(10) Pub. No.: US 2021/0107008 A1
(43) Pub. Date: Apr. 15, 2021

(54) System and method of isothermal DNA amplification devices with mobile devices for the cloud service

(71) Applicant: Genalyze LLC, Middletown, DE (US)

(72) Inventor: Chen-Yu Liu, Middletown, DE (US)

(73) Assignee: Genalyze LLC, Middletown, DE (US)

(21) Appl. No.: 17/067,559

(22) Filed: Oct. 9, 2020

Related U.S. Application Data
(60) Provisional application No. 62/913,164, filed on Oct. 9, 2019.
(60) Provisional application No. 63/089,539, filed on Oct. 8, 2020.

(51) Int. Cl. B01L 7/00

(52) U.S. Cl. CPC B01L 7/52; B01L 2200/0689; B01L 2300/1866; B01L 2300/1883

(57) ABSTRACT

A portable isothermal amplification device with a portable heat source and a thermal sensor linking to a cloud environment via mobile devices with a mobile application of the present invention for determining the DNA information of samples with a given primer in a manner of point-of-collection (in the field). The cloud environment with mobile devices can provide isothermal amplification information that may include isothermal amplification related data (e.g., genetic information of organism, cancer cells or viruses of interest), analysis methods, solutions provided by experts for access, storage, analysis and consulting. The information may also include gene expression levels of interest, DNA identity of samples, as well as treatment suggestion and professional lists for consulting. In addition, the cloud environment can provide further solution, which is based on the sequence presence information generated from the devices for the end users.

### PRIORITY CLAIM

This patent application claims priority to U.S. provisional application 62/913,164 filed on Oct. 9, 2019 and 63/089,539 filed on Oct. 8, 2020 the entire contents of which is incorporated herein.

### FIELD OF THE INVENTION

The present invention relates generally to a portable isothermal amplification device, method and system of isothermal amplification of nucleic acid in conjunction with a mobile device(s) for storing, analyzing, and/or accessing, and/or interpreting biological data that are associated with isothermal amplification, and providing a service in a cloud computing environment based on the biological data.

### BACKGROUND

Nucleic acid sequence identification is important for many applications, such as genetic study, inherited diseases, genealogy, pathogen identification, forensic, diagnostic, environmental monitoring and other applications. Isothermal amplification techniques have been used for nucleic acid sequence identification/quantification. Isothermal amplification techniques could be used to amplify the target nucleic acid of samples. Once target nucleic acid is amplified, a detection method to determine the amplified DNA quantity will be employed. For example, one of the detection methods is to determine the change in color or fluorescence intensity due to increasing number of dye molecules interacting with the amplified DNA or amplification of DNA changes the pH value of reaction which can be detected by a pH indicator. While sequencing techniques may be used for nucleic acid sequences information, an isothermal amplification method determines the amount of amplified target nucleic acid with a pre-designed sets of primers.

A number of alternative isothermal amplification techniques are presently under investigation and development. Isothermal amplification techniques usually are carried out with pre-designed sets of primers. The pre-designed set of primers are permitted or encouraged to bind to the template of genetic material, and nucleic acid polymerase and nucleotides are introduced for template DNA amplification at a constant temperature or within a narrow range of temperature.

Some of the isothermal amplification techniques require much less resource and are less sensitive to the reaction conditions while comparing to other techniques such as polymerase chain reaction (PCR). Many isothermal amplification techniques have been applied in a manner of point-of-collection. In addition, the reagents for certain nucleic acid isothermal amplification techniques could be lyophilized into powder, which allows the better preservation and easy to use (e.g. “Stable reagents and kits useful in loop-mediated isothermal amplification (LAMP)”, US patent application number US20080182312A1).

The further combination of isothermal amplification techniques and mobile devices allow users to obtain species' or strains' information from samples in a timely manner. Also, this combination will reduce the possibility of nucleic acid degradation of sample during transportation.

For example, the nucleic acid isothermal amplification techniques with a mobile device has been used to determine presence of some particular nucleic acid sequences in biological samples in a point of collection manner (“A portable device for nucleic acid quantification powered by sunlight, a flame or electricity”, Nature Biomedical Engineering, vol 2, 2018, p 657-665). On the other hand, the device in the reference mentioned above is still more expensive and complicated in design. To have a more simple and inexpensive device for isothermal amplification at home or field is desired. One of essential parts of isothermal amplification device is the heat source, which maintains the isothermal amplification reaction to occur within a range of temperature. There are many isothermal amplification devices which still require clunky heat sources or the heat sources are not re-usable. Therefore, it would be desirable for a convenient and portable heat source which allow users to access and conduct isothermal amplification easily. Furthermore, it would also be desirable to have an easy way to monitor the temperature of the isothermal amplification reaction without using an electronic sensor to ensure the reaction is conducted at a suitable temperature.

Given the fact—isothermal amplification techniques with mobile devices can be applied in a location with limited material resources or in a manner of point-of-collection. However, it still requires experts or trained personnel on the particular field to conduct the procedures for isothermal amplification of nucleic acids, and/or to interpret the results and/or provide solutions or advices based on the results generated from isothermal amplification techniques. For example, some of procedures, for conducting isothermal amplification techniques, require users to have special knowledge to handle the samples and be familiar with certain protocols. Also, the results obtained from isothermal amplification usually provide the information related to the presence or the amount of some particular nucleic acid sequences in biological samples only. This information also associates with organisms. These organisms carry the nucleic acid sequences and/or gene expression levels of certain genes from the organisms. The type of the information usually needs further interpretations by an expert who is familiar with the information. Or the information requires complicated algorithms and/or databases for further interpretation, which is difficult to carry out in a point-of-collection manner. As of 2020, there was a SARS2-COVID pandemic and many techniques have been applied in detecting the virus. Nucleic acid detection is the most accurate among other approaches. However, there were a limited number of methods that integrate the disease control and the information of disease spread in a real-time manner. Therefore, there is an urgent need for a method to resolve the information gap between the usage and collection of nucleic acid information of a virus.

Furthermore, even when said information could be interpreted correctly, under certain situation, it still requires at least an expert's advice to address an end user's problems because end users may not have the proper knowledge or resources to address their problems.

For example, while a technician may be able to perform isothermal amplification of nucleic acid for test subjects, he/she may not have enough resource to identify the subjects without accessing a database or consult other experts on some specific applications. For example, in an outbreak of African swine fever, to evaluate the severity of this infection disease, it requires performing many tests in different localities and times. Thus, the information is collected by many people at many locations. The information of whether a particular pig is infected may combine with the pig's status such being asymptomatic or death, and is further used as a parameter for constructing a prediction model of African swine fever distribution as taught in the reference (“A Mathematical Model that Simulates Control Options for African Swine Fever Virus (ASFV), PLoS One. 2016; 11(7): e0158658). The model provides the measure options for controlling the disease distribution. However, methods used for collecting infected pigs' information in the outbreak are still difficult to carry out in a manner of point-of-collection.

While recent technology development for point-of-collection has advanced, most of the developments only address the issues of limited resources on physical or hardware aspects.

Furthermore, for general users, they may not even have information about which experts or specialists they can consult with.

In addition, there are various types of pre-designed primer sets, as well as various protocols and devices available for application of isothermal techniques. However, there is limited information for usage of these protocols, devices and pre-designed primer sets in term of sharing and evaluation as well as instructions.

Furthermore, the isothermal amplification related data obtained from point-of-collection may be fragmented as well as the information for inventory of kits, reagents, cartridges and devices for isothermal amplification techniques. The information may not be convenient to organize, store, manage and access.

It would be desirable to have a method that can provide an advice/solution based on the presence or quantity of certain nucleic acids with specific sequences from biological samples. This is obtained via isothermal amplification techniques in a point-of-collection manner with mobile device(s).

Also, there exists a need for a method that instructs users, shares, analyzes and evaluates various primer sets or protocols or analysis methods that use isothermal amplification techniques in a point-of-collection manner.

Furthermore, there is a need for a management platform associated with an isothermal amplification technique. Thereby, said management platform keeps and organizes the inventory of kits, reagents, cartridges and devices, as well as isothermal amplification data.

### SUMMARY

The present invention advantageously fills the aforementioned deficiencies by providing an apparatus, system and a method of isothermal DNA amplification with a mobile device.

The present invention comprises an apparatus for isothermal nucleic acid amplification with mobile devices, as well as a novel approach for storing, analyzing, and/or accessing, and/or interpreting biological data that are associated with isothermal amplification, and providing a service based on the biological data, in a cloud computing environment.

The present invention further comprises techniques to share, store, analyze, interpret biological related information (e.g., biological data, protocols, analysis methods, etc.) obtained from isothermal amplification of nucleic acids techniques and a mobile device to provide isothermal nucleic acid amplification in a point-of-collection manner.

Furthermore, based on said biological related information, experts can provide advices, solutions and information to the users of isothermal nucleic acid amplification technology. The isothermal nucleic acid amplification is carried out in a point-of-collection manner with mobile devices.

The present disclosure also provides a system of isothermal amplification technique that provides a convenient heat source and a simple temperature sensor, to maintain and monitor the temperature of an isothermal amplification reaction using or with mobile devices in a point-of-collection manner.

The present disclosure also provides a novel method for sample extraction, sample preparation, conducting isothermal amplification, shifting or distributing various information generated by or related to isothermal amplification of nucleic acid techniques from mobile devices, isothermal amplification devices and/or sample preparation devices (e.g., test subjects' identification, protocols, analysis methods, sample preparation data, sequences information of primer sets, etc.) to a cloud-based network (e.g., a local cloud or a remote cloud) in a point-of-collection manner.

In one embodiment, the isothermal amplification device comprises a test kit and a heat source for target nucleic acid sequence amplification.

In one embodiment, the test kit comprises a cartridge, wherein the isothermal nucleic acid reaction is held, and the adjacent heat source has heat communication with said cartridge.

In one embodiment, the reaction chamber of a cartridge is a sample well that holds all reagents required for isothermal nucleic acid amplification, wherein the reaction chamber is of interest area for colorimetric detection.

In one embodiment, the test kit comprises, but is not limited to, DNA polymerase and/or reverse transcriptase, nucleotide, reaction buffers and nucleic acid primers for target nucleic acid. The primers are used to amplify the target nucleic acid.

In one embodiment, dye molecules are used as an indicator for amplified DNA.

In one embodiment, the test kit may comprise negative control samples and/or positive control samples for the target nucleic acids. In the negative control samples, there is no target nucleic acid; while in the positive control samples, the target nucleic acids may be amplified. The positive control samples will use the same reagents and primers as the test subject's samples use, and the isothermal amplification reaction is carried out under the same condition for positive control samples and test subject's samples. Therefore, it helps users to determine if isothermal amplification has been carried out properly. And/or the test kit may comprise negative control samples. The negative control samples do not contain any nucleic acid with target sequence. The negative control samples will use the same reagents with the test subject's samples' and the isothermal amplification reaction is also carried out under the same condition for both negative control samples and test subject's samples. Therefore, usage of negative control samples allows users to determine if their reagents are contaminated with target nucleic acid.

In one embodiment, a test kit can be lyophilized and have a longer storage time.

In one exemplary embodiment, a sample preparation device comprises a kit cartridge and/or a heat source. Said cartridge may contain reagents for sample extraction or preparation.

In one embodiment, the heat source comprises a thermal storage medium in thermal communication with a heating material. The thermal storage medium comprises a phase changing material (PCM). The heating material and thermal storage medium are configured to maintain the temperature of isothermal amplification reaction or sample preparation in a cartridge adjacent to the heat source.

The utility of this integrated, the test kit is demonstrated by amplifying target DNA with the isothermal amplification reaction. The test kit is particularly suitable for use in the field, in resource-poor regions of the world (where funds and trained personnel are in short supply), in remote areas, and at home. Other heat sources, such as battery-powered sources, solar-powered sources, and other heat sources that derive heat from exothermic reactions are all suitable.

The heat source or heating material (described in more detail below) is suitably in thermal communication with the chamber of a cartridge and/or a cartridge of a test kit, a fluidic element, or any combination thereof.

In other embodiments, a conductive material (e.g., metal) places the heating material in thermal communication with the cartridge of a test kit or other component of the system. The isothermal amplification device may also include one or more manually-operated elements that allow the user to place various components of the device into fluid and/or thermal contact with one another.

As discussed elsewhere herein, the heat source or heating material, once activated or heated up, serves to supply heat to the cartridge of a test kit or other element. In this manner, a device can be constructed that is capable of performing a reaction or other process that requires heat, while the PCM stores and regulates the release of heat.

In some embodiments, the heating material is fluid.

In one embodiment, an amount of fluid (e.g., water) is packaged with the device or added by user with a specified amount of fluid so that the fluid is available to the device at the time of activation or heating up.

The isothermal amplification or sample preparation devices or apparatuses may include a cartridge that is an enzymatic reaction chamber. Such chambers may be adapted for isothermal amplification or sample preparation. In one of embodiments, the chamber may include one or more pre-stored (e.g., dried) reagents within. The chamber may also be an incubation chamber, or even a culture growth chamber.

A variety of fluidic elements may be present. Such elements may be valves, pistons, membranes, cantilevers, and the like.

In this way, the heating material or heat source may be configured so as to supply sufficient heat to the enzymatic reaction chamber or cartridge. Thermal storage media used in the disclosed devices may include a wax, a thermoplastic, a salt hydrate, a fatty acid, a fatty acid ester, or any combination thereof. Paraffin wax (detailed elsewhere herein) is considered a particularly suitable thermal storage medium. The thermal storage medium is suitably a material—such as wax that undergoes a phase change at a particular temperature. Such materials permit the construction of devices that controllably maintain a particular temperature range, which is regulated by the phase change (e.g., melting) temperature of the thermal storage material.

Various materials may be used as heating materials; materials that are chemically reactive are considered suitable. Such materials include magnesium-iron alloy, calcium oxide, sodium acetate, potassium permanganate (reactive with glycerol), and the like. Materials that are inert chemically are also considered suitable. Such material includes water, beverage, oil, wax and the like.

In one of embodiment, these methods suitably include contacting a chemically-reactive heat source with a fluid so as to generate heat; the chemically-reactive heat source being in thermal communication with a thermal (e.g., heat) storage medium.

In one exemplary embodiment, the methods of processing a sample comprising contacting a chemical-reactive heat source with a fluid so as to generate heat; the chemical reactive heat source being in thermal communication with a thermal storage medium (which may also be referred to as a heat sink), the chemical-reactive heat source and thermal storage medium being configured to maintain the temperature range of a sample in a cartridge adjacent to the heat source.

In one embodiment, the methods may include heating up a chemically inner heating material by physical methods such as using a microwave oven, an oven or a stove. Said heating material contacts with the thermal storage medium of a sample preparation or isothermal amplification device.

In one embodiment, said heat source or heating material can be heated up by a microwave oven, a baking oven, a stove or other heating equipment.

In one exemplary embodiment, the methods of processing a sample comprise a fluid heat source so as to conduct heat; the a fluid heat source being in thermal communication with a thermal storage medium (which may also be referred to as a heat sink), the a fluid heat source and thermal storage medium being configured to maintain the temperature of a sample in a cartridge/reaction chamber adjacent to the heat source.

In one exemplary embodiment, an isothermal amplification or sample preparation device of the system includes a water-thermostat container and an isothermal nucleic acid amplification cartridge/kit. Said device does not require any external instrument and/or power. Said cartridge/kit is directly powered by high temperature fluid and the temperature is regulated by a PCM. The high temperature fluid is heated up with means including but not limited by a microwave oven, an oven or a stove. And said fluid may contain water. Thereby, said container water-thermostat container may be reused.

In one embodiment, a temperature indicating label may contact with the cartridge or reaction chamber to ensure the isothermal amplification reaction is conducted within a suitable temperature range.

In one embodiment, a temperature indicating label may contact a heating material to ensure the heat source reaches a desire temperature. Once the desire temperature is reached, said temperature indicating label may change its color or form.

In one exemplary embodiment, water is used as a heating material. A temperature indicating label contacts with said water and said water is heated up in an oven or microwave oven till the temperature indicating label changes of its color. Thereby, the users are able to tell if said water reaches the desired temperature by the change of temperature indicated label's color.

Said temperature indicating label may include but is not limited to liquid crystal temperature label, temperature crayon, temperature indicating liquids and paints.

In one embodiment, high temperature water is added into an isothermal amplification or sample preparation device of the system, and the reaction chamber or cartridge's temperature is regulated and rendered independent of ambient temperatures with the aid of a phase change material.

In one embodiment, a PTC heating element is used as a heat source and used to maintain isothermal nucleic acid amplification reaction.

In one embodiment, the heat source comprises a heating element and/or an electrical thermostat device.

In one embodiment, the heat source and thermal storage medium may be configured to maintain the temperature of a sample nucleic acid in a reaction chamber of a cartridge adjacent to the heat source at a temperature in the range of from about 25 deg. C. to about 80 deg. C. for a period of time from about 5 minutes to about 120 minutes in an environment of ambient temperature ranging from 0 deg. to 50 deg. C. As used herein, the term “about” refers to plus or minus 10 percent of the stated amount.

It should be understood that the term “adjacent” does not require that the reaction vessel be in direct contact with the heat source.

The system can include a cloud computing environment in communication with multiple mobile devices while said mobile devices are linked to isothermal amplification devices (or sample amplification module) or a signal detecting modules. One unlimited example of said isothermal amplification devices is a mobile device with a heat source and a cartridge.

The cloud computing environment can include at least one server. The at least one server can be configured to communicate with at least one of the mobile devices and at least one of the mobile devices is remote from the at least one server to receive and store isothermal amplification information from the at least one mobile device once the isothermal amplification related data are generated.

Embodiments of the present techniques are described herein by reference to isothermal amplification information generated/transmitted by a mobile device with isothermal amplification devices (or sample processing module) or signal detecting modules which quantifies amplified nucleic acid from isothermal amplification of test subjects' target nucleic acid. One not limited examples of said signal detecting modules is a mobile device.

Embodiments of the present techniques are described herein by reference to sample preparation data generated by a sample preparation device, isothermal amplification related data generated by a mobile device and isothermal amplification devices (sample processing module) or/and signal detection modules.

The method of present disclosure suitably includes performing isothermal nucleic acid amplification of target nucleic acid, e.g., loop-mediated amplification (LAMP), and the like in a manner of point-of-collection.

In one of embodiments, the DNA/RNA for isothermal nucleic amplification reaction is extracted and prepared by a sample preparation device from any fluid or tissue of a test subject.

In one of embodiments, isothermal amplification device dispenses required reagents, enzymes, nucleotides, primers and samples into the reaction chamber.

In one embodiment, the system comprises a cloud computer, a mobile device, an isothermal amplification device and a test kit.

The methods may include detecting the presence of one or more target nucleic acids following amplification of the sample's nucleic acids.

In one embodiment, said mobile device involves imaging a colorimetric change in a nucleic acid isothermal amplification reaction of a sample or processing sequencing data from a portable nucleic acid sequencer. The mobile device includes an application installed on or accessible (e.g. the internet) and a mobile device accessory or signal detection module.

Furthermore, based on the information of presence of target sequence in the sample and/or other related information, experts or the system can provide advice and/or solutions (i.e. procedures, warnings) to the clients.

For example, the techniques relate to a cloud computing environment configured to receive the information from one or more mobile devices linked to at least one isothermal amplification device. Said isothermal amplification device with a mobile device can be used to quantify amplified nucleic acid from at least one test subject in a manner of point-of-collection.

In certain embodiments, the information may be stored and/or analyzed using the cloud computing environment, which may reduce the processing and/or storage burden associated with the mobile device. Storage of information in a cloud computing environment as provided herein allows location-independent access and storage, as well as backup storage. In addition, the information on cloud may enable online experts to find the solution or provide advices for clients without limitation of locations. And the processed information, advices (i.e. a list of experts or specialists to consult with for particular applications) or solutions can be accessed on the cloud environment without too many physical limitations.

The cloud computing environment may also provide sharing of protocols, instructions to perform isothermal amplification, analysis methods, pre-designed primer sequence information as well as analysis, reporting, advices and/or solutions. Thus, users will not need any particular training or education background could also use isothermal amplification techniques in a manner of point of collection.

In addition, the sharing and distributed processing also allows computing and/or storage resources to be allocated (e.g., crowd-sourced) to particular projects, clients or experts within the cloud environment. Such an implementation may allow clients or experts to access the information and advanced data processing platform. Additionally, based on the shared information, experts may provide further solution to clients while said experts could also be the users of information.

Alternatively or additionally, such an implementation can provide a convenient venue or portal for purchasing a product from a supplier of a component of the isothermal amplification techniques (e.g., sample preparation cartridge, isothermal amplification kits or pre-designed primer sets).

Alternatively or additionally, the information from test subjects of isothermal amplification techniques could be used for appraisement. A supplier of test subjects can provide the information through the portal to potential buyers. For example, a biological produce may be tested by isothermal amplification technique to identify the source of origin.

Alternatively or additionally, in one embodiment, the isothermal amplification information of test subjects can be a product. For example, the isothermal amplification information may be some particular Single-nucleotide polymorphisms (SNPs) of test subjects. And the SNPs may be associated with certain phenotypes.

Additionally or alternatively, users may purchase products from suppliers based on the isothermal amplification related data of products, which is processed or stored on the cloud environment. Herein, the products are the test subjects.

Additionally or alternatively, users may purchase service from experts for advices or procedures as well as data analysis reports and/or software based on the isothermal amplification related data from at least one test subject.

Additionally or alternatively, users may purchase or use isothermal amplification related data that is associated with test subject's samples for further analysis or prediction.

The cloud computing environment may also facilitate a virtual plug and play interaction between mobile devices and data analysis platforms. Users may relinquish responsibility for servicing and updating dedicated programs for analyzing isothermal amplification data, because maintenance of the data analysis software is conducted via the cloud monitoring systems. Such an arrangement frees up IT resources at the user or client site.

The cloud computing environment may also promote the development and sharing of isothermal amplification related data, customizable protocols, pre-designed primer set, data generated from isothermal amplification as well as data analysis software and methods.

Also, based on the purpose for the usage of isothermal amplification kit, the user develops a customized DNA primer set or combination of enzymes and reagents. The protocol or sequences of primers may be used to drive the sample preparation and/or isothermal amplification devices or/and mobile devices to perform each of the required steps (e.g., primer sequence information for a particular organism, mixing, incubation, splitting of samples and reagents, etc.). The customized protocol (e.g., optimized protocol) and/or primer sets sequence information and/or corresponding analysis methods may be submitted to the cloud computing environment for other users to use.

Additionally, based on the purpose for the usage of isothermal amplification information, the user develops analysis methods and/or data processing workflow and/or solutions. The corresponding analysis methods and/or data processing workflow and/or solutions may be submitted to the cloud computing environment for other experts or users to use.

In addition, the cloud computing environment enables the use of particular protocols and/or primer sets and services (e.g., by requesters or citations in publications), the rating of protocols and/or primer sets and/or services and/or experts, and certification of the protocols and/or primer sets and/or services and/or experts.

Indeed, application-specific components of the isothermal amplification techniques (e.g. cartridges/devices and/or primer sets) may be developed by their suppliers based in part on the reception of the submitted isothermal amplification information (protocols and/or primer sets and/or analysis methods).

To further promote the development and sharing of isothermal amplification information (e.g., isothermal amplification data, protocols, primer sets, analysis methods, solutions), the submitter of isothermal amplification information may be credited with credit to purchase products from the supplier or receive a fee. Thus, the cloud computing environment provides a platform for the sharing isothermal amplification information and development of protocols, primer sets, analysis methods, as well as providing solutions for users' particular problems when using with the products from suppliers (e.g., generic cartridge, primer sets, devices, isothermal amplification related data).

Furthermore, the cloud computing environment enables the use of particular methods or solutions (e.g., by requesters or citations in publications), the rating of methods or solutions, and certification of the methods or solutions.

Indeed, application-specific methods or solutions may be developed by the supplier of a product (i.e. generic cartridge, produce, food, service) based in part on the reception of submitted data and/or protocols and/or primer sets.

Alternatively or additionally, application-specific methods or solutions may be developed by the crow-source in part on the reception of submitted isothermal amplification information (i.e. data, protocols, primer sets, analysis methods and solutions).

The system can include a cloud computing environment in communication with multiple computer systems. The cloud computing environment can include at least one server and at least one processor.

The at least one server can be configured to communicate with at least one of the computer systems to receive and store isothermal amplification related data (i.e., test subjects' manifest, amplification data, protocols in isothermal amplification, primer sets for isothermal amplification, sample preparation data, services and advices from experts).

The at least one processor can be configured to monitor a request by a requester for the isothermal amplification information and to credit a submitter of the isothermal amplification information with credit for purchasing product (i.e., consumables of isothermal amplification technique) or a fee from a supplier of product for each request for the isothermal amplification information.

Based on particular purposes, the cloud computing environment may monitor requests and usage details as well as inventory of kits, cartridges and provide suggestion and notification for more suitable kits, primer sets or cartridges for future usages.

The present disclosure still further provides a computer-implemented method for analyzing biological samples in a cloud computing environment. The method can include receiving, at a server, sample preparation and/or isothermal amplification data and generating, via a processor, a sample preparation and/or isothermal amplification data log based at least on the sample preparation and/or isothermal amplification related data.

The method can include receiving, at a server, isothermal amplification data and generating, via a processor, data analysis report based at least on the isothermal amplification data.

The method can also include receiving, at the server, isothermal amplification data and generating, via the processor, a summary of amplification nucleic acid targets based at least on the isothermal amplification data and the specific analysis approach.

The method can further include receiving, at the server, a summary of amplified nucleic acid targets and generating, via at least one expert, a report or advice or solution based at least on the summary of amplified nucleic acid targets.

The isothermal amplification of nucleic acid technique uses pre-design primer sets to determine the presence or amount of target nucleic acid from a biological sample. Because the pre-design primer sets have already decided the sequences of target nucleic acid which is from a biological sample, the nucleic acid sequence of reaction product is determined. The present disclosure provides a method using a sequencer as a sample signal processing module instead of merely using the sequence information from sequencer to identify an organism or virus from a sample. In other words, the present disclosure method uses the amount information of amplified nucleic acid to determine the present of a target sequence instead of merely retrieve sequence information from amplified nucleic acid.

The disclosure is not, however, limited by the advantages of the aforementioned embodiment. The present techniques may alternatively or additionally be applied to devices capable of generating other types of isothermal amplification related data, such as nanopore sequencing data. Nanopore sequencer can be regarded as a signal detection module. The nanopore sequencing data may be used to quantify the amount of target nucleic acid via isothermal amplification of nucleic acid amplification. The isothermal amplification data generated by nanopore sequencer may be stored, processed, and/or accessed, and/or analyzed and interpreted by users or experts for providing advices or solutions in conjunction with the cloud computing environment as provided herein.

In one or more embodiments, the present invention further comprises non-transitory computer readable memory devices (e.g. smartphone, tablet, etc. with mobile application installed or accessible thereon, a computer co-located with the separation preparation device and/or the isothermal amplification device) including computer-readable instructions configured to instruct a computerized system to perform one or more methods disclosed herein.

In one or more embodiments, the present invention further comprises a computer with non-volatile memory including computer-readable instructions configured to instruct a computerized system to perform one or more methods disclosed herein.

In one or more embodiments, the present invention further comprises a computer program product comprising a non-transitory computer readable medium storing a plurality of instructions that when executed control a computer system to perform the one or more methods disclosed herein.

In one or more embodiments, the present invention further comprises a computer program product comprising, a computer with a computer with non-volatile memory and readable medium storing a plurality of instructions that when executed control a computer system to perform the one or more methods disclosed herein.

In one or more embodiments, the present invention further comprises a computer system comprising one or more processors, wherein: either: the computer system further comprises a computer readable medium storing a plurality of instructions, or the one or more processors include circuitry configured to execute the plurality of instructions; for performing the one or more methods disclosed herein (e.g. FIG. 6—sharing and monitoring the popularity of isothermal amplification information via the cloud computing environment; FIG. 7—the major steps involved in a typical isothermal amplification information analysis workflow; and FIG. 8—a method of interaction of a user and devices with the cloud-based computing environment.).

### BRIEF DESCRIPTION OF THE DRAWINGS

FIG. 1 is a diagrammatical overview for a system incorporating a cloud computing environment in accordance with the present disclosure.

FIG. 2 is a diagrammatical overview of an individual node 29 of the cloud computing environment of the type discussed with reference to FIG. 1.

FIG. 3 is a diagrammatical overview of a sample processing module (isothermal amplification cartridge) 37 with mobile device 41 that may be used in conjunction with the cloud computing environment of the type discussed with reference to FIG. 1.

FIG. 4 is a diagrammatical overview of a sample preparation device that may be used in conjunction with the cloud computing environment of the type discussed with reference to FIG. 1.

FIG. 5 is a schematic overview of a cloud-based computing environment that enables sharing of isothermal information (e.g., sample preparation protocols/isothermal amplification related data, analysis, advices and solutions of experts) and popularity monitoring.

FIG. 6 is a flow diagram of a method of interaction of submitters, requesters, and a supplier with respect to the sharing and monitoring of the data, protocols, analysis methods, advices and solutions on the cloud-based computing environment of the type discussed with reference to FIGS. 1 and 5.

FIG. 7 is a schematic overview of a cloud-based computing environment to facilitate a cloud-guided isothermal amplification information analysis workflow.

FIG. 8 is a schematic overview of a flow diagram of a method of interaction of a user and devices with the cloud-based computing environment of the type discussed with reference to FIGS. 1 and 7.

FIGS. 9A and 9B are depictions an isothermal amplification device of the present invention. FIG. 9A is a longitudinal cross-sectional view of a thermal container with a cartridge positioned within and near the top.

FIG. 9B is an exploded view of the thermal container with the cartridge removed and links to a mobile device.

FIG. 10 is the chart of temperature versus time after the isothermal amplification device is heated up or activated by hot water.

### DETAILED DESCRIPTION OF THE INVENTION

### Glossary of Terms

As used herein, the term “isothermal amplification of nucleic acid techniques” or “isothermal amplification techniques” or “isothermal amplification” or “nucleic acid isothermal amplification” can refer to the nucleic acid amplification technique carried out at a constant temperature. A list of the techniques include but is not limited to: transcription mediated amplification (TMA) or self-sustained sequence replication (3SR), nucleic acid sequence-based amplification (NASBA), signal mediated amplification of RNA technology (SMART), strand displacement amplification (SDA), rolling circle amplification (RCA), loop-mediated isothermal amplification of DNA (LAMP), isothermal multiple displacement, amplification (IMDA), helicase-dependent amplification (HDA), single primer isothermal amplification (SPIA), and circular helicase dependent amplification (cHDA), recombinase polymerase amplification (RPA) (NUCLEIC ACID ISOTHERMAL AMPLIFICATION TECHNOLOGIES A REVIEW, Nucleosides, Nucleotides, and Nucleic Acids, 27, 224-243, 2008). Nonlimiting examples are also described in (Isothermal Amplification of Nucleic Acids, Chem. Rev. 2015, 115, 12491-12545, Isothermal Amplification Methods for the Detection of Nucleic Acids in Microfluidic Devices, Biosensors, 3, 18-43, 2013); or isothermal amplification techniques combining with other molecular biology complements such as CRISPR. One of the examples is CRISPR-Cas9-triggered nicking endonuclease-mediated SDA method (CRISDA). Furthermore, there is another example as taught in one recent study. (Chem. Commun., 2020, 56, 3536-3538)

As used herein, the term “isothermal amplification related data” refers to but is not limited to: test subjects' manifest, amplification data, protocols in isothermal amplification, primer sets for isothermal amplification, sample preparation data, isothermal amplification signals or processed signals, information of organism, cancer cells or viruses of interest.

When referring to isothermal amplification data, it is provided in connection with determining the amplification signals of isothermal amplification reaction of subjects' nucleic acids. For example, isothermal amplification data may include, but is not limited to, the presence or quantity of prior sequence or consensus sequence information from a test subject.

As used herein, the term “isothermal amplification information” refers to isothermal amplification data, protocols and/or primer sets and/or analysis methods and/or solutions.

Alternatively, said isothermal amplification information may include the sequences of primer sets used in isothermal amplification of nucleic acid.

Alternatively, said isothermal amplification information may include the protocols used in isothermal amplification of nucleic acid.

Alternatively, said isothermal amplification information may include the analysis methods for analyzing isothermal amplification data.

Alternatively, said isothermal amplification information may include solution based on analysis or interpretation of isothermal amplification data.

Alternatively, said isothermal amplification information may include data associated with test subjects of isothermal amplification techniques such as manifest. For example, certain portions of a test subject's nucleic acid sequence may be unique and may be used to identify the test subject. Thus, isothermal amplification information may include the identity of a test subject obtained from the isothermal amplification technique.

Alternatively, said isothermal amplification information may include the sample preparation related data or any data used in sample preparation.

Isothermal amplification information can be provided, for example, by a user, a third party, a mobile device, an isothermal amplification device, and/or a sample preparation device.

In some embodiments, isothermal amplification information may be provided by a supplier of predesigned primer sets to a sample preparation device or an isothermal amplification device or mobile device. In some embodiments, isothermal amplification information is provided by a user to a third party, or to a cloud computing environment. In some embodiments, isothermal amplification information is provided from a cloud computing environment or a third party to a mobile device.

As used herein, the term “sample preparation related data” refers to information related to a sample preparation/extraction procedure, including executable instructions for carrying out a sample preparation/extraction procedure on a device, and/or data related to a specific sample preparation/extraction procedure such as sample identification, date, time, location, temperature and other particular details of sample preparation procedure or source. For example, sample preparation related data can include sample extraction, sample preparation recipe/protocol identification, sample preparation cartridge identification, cartridge preparation identification, sample extraction instrument identification, sample preparation instrument identification, and other parameters. In some embodiments, sample preparation related data is input or provided by a user to a sample preparation device. In some embodiments, sample preparation related data is provided by a user to a third party, or to a cloud computing environment. In some embodiments, sample preparation related data is provided from a cloud computing environment or a third party to a sample preparation device. In some embodiments, sample preparation includes nucleic acid extraction from a sample.

As used herein, the term “pre-designed primer” or “primer set” refers to an oligonucleotide which is complementary to its target nucleic acid sequence and used for nucleic acid amplification. Said sequence of target nucleic acid is prior known or consensus sequence.

As used herein, the term “mobile device” means a mobile apparatus comprising a processor, memory, input/output functions, display, internet access, etc. that is capable of running a programmed application suitable for executing the embodied functionality while suitable traditional Smartphone or Smartphone-type devices may include but are not limited to, e.g., products such as the iPhone (Apple, Inc.) or Android (Google Inc.)-based Smartphones and tablet computers.

As used herein, the term “experts” could include but is not limited to trained specialists or people who have certain specific knowledge and are capable to interpret the data or analysis obtained from isothermal amplification techniques. A list of experts could include but is not limited to a medical doctor, a genetic consultor, a microbiologist, a virologist, a veterinarian, a biologist and health professional. It could also be artificial intelligence or a program.

As used herein, a professional list is a list that includes experts or specialists.

As used herein, the term “point-of-collection” is defined as a testing at or near the point of sample collection—that is, at the time and place of sample being collected.

As used herein, the term “test subject” may include but is not limited to a microbe, plant, animal, human, soil, food, environment with microbes or materials contain with nucleic acids. For example, a test subject could be the meat collected from a restaurant or the water collected from sewerage.

As used herein, “advice” may refer to but is not limited to an advice, an evaluation or prediction, status of real time environment monitoring or diagnosis of at least one test subject. Alternatively, “advice” may also refer to guidance or recommendations offered with regard to future action.

As used herein, the term “isothermal amplification cartridge” or “isothermal amplification device” refers to a device which can hold a sample and reagents, and which may provide one or more chambers for isothermal amplification. In some embodiments, isothermal amplification devices may include a signal detection module (e.g. colorimetry, fluorometer, turbidimeter, electrical conductivity meter, nanopore sequencer) or a sample amplification module. In some embodiment, the sample amplification device may contain a cartridge that can be disposable and hold the isothermal amplification reagent.

As used herein, the term “protocol” refers to a method, step or instruction or set of methods, steps or instructions performed in completing a task, such as preparing nucleic acid samples. To complete a task, a sample preparation protocol typically includes a step-by-step set of instructions. The protocol may contain only a sub-set of the necessary steps to complete the task. The set of instructions can be performed entirely in a manual manner or in different level of automation manner. The protocol may include interactive instructions from a device. For example, a sample preparation protocol may have an initial step and is shown on the display of a device, instructing the manual introduction of a nucleic acid sample or cell lysate into an inlet port of a sample preparation cartridge, and introduction of other reagents with specified quantities into the other inlet port of the same sample preparation cartridge. The sample preparation device may automatically mix the sample and reagents, and then separate the nucleic acids from the sample.

As used herein, the term “sample preparation” refers to ways in which a sample is processed. In typical embodiments, sample preparation occurs before analysis of the sample. However, sample preparation may occur before, during, or after performance of one or more analyses of the sample. In some embodiments, sample preparation may include, but is not limited to, one or more of extracting, purifying, separating, or combining samples. The extracting, purifying, separating or combining may be partial or full extraction, purification, separation or combination. In some embodiments, sample preparation may include, but is not limited to, lysis, cleaving, degrading, annealing, hybridizing, denaturing, and other samples to process a sample. Any suitable sample preparation technique as known in the art may be used in the protocols, methods and devices presented by reference (e.g. see: Michael R. Green et al., Molecular Cloning: A Laboratory Manual, 4th Edition, 2012.)

As used herein, the term “sample preparation device” refers to a device/cartridge which can hold a sample and reagents, and that provides one or more chambers to convert samples into a proper form for isothermal amplification of nucleic acids.

The term “generic cartridge” refers to a sample preparation cartridge or isothermal amplification cartridge which is not limited to any one particular protocol. For example, in some embodiments, a generic sample preparation cartridge or isothermal amplification cartridge may not include any reagents, and reagents are added to the cartridge as needed by the user. In other embodiments, a generic cartridge or isothermal amplification cartridge may include specific reagents, compartments and connections required for and dedicated to a specific application (e.g., plant seed sample preparation). The use of the generic cartridge enables a user to utilize their own customized protocol for use with the cartridge to address the specific need or application of the user.

As used herein, the term “isothermal amplification kits” refer to reagents for isothermal amplification of nucleic acid reaction. For example, in some embodiments, an isothermal amplification kit may include DNA polymerase, nucleotides and buffers.

The term “generic cartridge” refers to a sample preparation/isothermal amplification cartridge, which is not limited to any one particular protocol. For example, in some embodiments, a generic sample preparation cartridge/isothermal amplification cartridge may not include any reagents, and reagents are added to the cartridge as needed by the user. In other embodiments, a generic cartridge may include specific reagents, compartments and connections required for and dedicated to a specific application (e.g., Kaposi sarcoma sample preparation). The use of the generic cartridge enables a user to utilize their own customized protocol for use with the cartridge to address the specific need or application of the user.

As used herein, the term “certified status” refers to a designation that can be conferred to a protocol when one or more criteria have been met. For example, a protocol can achieve certified status based on input from other users in the form of a rating system or other peer-approval process. Alternatively or additionally, a protocol can achieve certified status based upon one or more publications where the protocol is noted, followed and/or discussed by the authors. A protocol that has achieved certified status may also encourage more users to use a particular protocol.

As used herein, “signal detection module” is a module determining the signal of isothermal amplification of nucleic acids, and includes all the hardware and software required for such.

As used herein, “sample amplification module” is a module processing isothermal amplification of nucleic acids, and includes all the hardware and software required for such.

As used herein, “isothermal amplification signals” is the signal of isothermal amplification of nucleic acids. That may include but not limited to fluorescence emission from chelated dye molecule or voltage change between a membrane of a nanopore device or translucence of emission light.

As used herein, the term “crowd-sourced” refers to computing resources allocated to particular projects or users within the cloud computing environment. One example of crowdsourcing in the methods provided herein includes analysis and/or interpretation of isothermal amplification data and/or further provide reporting and/or solution.

As used herein, the term “solution” includes but not limited to treatment, procedures, information, communication.

In one embodiment, the experts or specialists are among breeders, veterinarians or geneticists, the SNPs information could be breed-specific polymorphisms obtained from test subjects. The SNPs information of test subjects could be distributed to internet and analyzed for identification of breeds.

As used herein, the term “origin information” refers to the biological source of a test subject or phylogenetic relatedness of a test subject with others from a database.

In one embodiment, the SNPs information may be related to some phenotypes or diseases.

In one embodiment, the SNPs information includes but is not limited to any allele information listed in (i.e. NCBI database ClinVar)

The present invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

As used herein, the term “link” is defined as associate or connect. A component of the system may be connect or associate to other component in order to perform the function of system. For example, “link” may two components connected with a USB cable or wirelessly with a Bluetooth, or the reaction chamber of a cartridge with a camera.

As used herein, the term “manifest” refers to the information or data associate with a sample prior to carrying out isothermal amplification process. The non-limiting examples of a manifest include the amount of sample, location of sample collection, ID of a sample, the owner of a sample, the status of a sample.

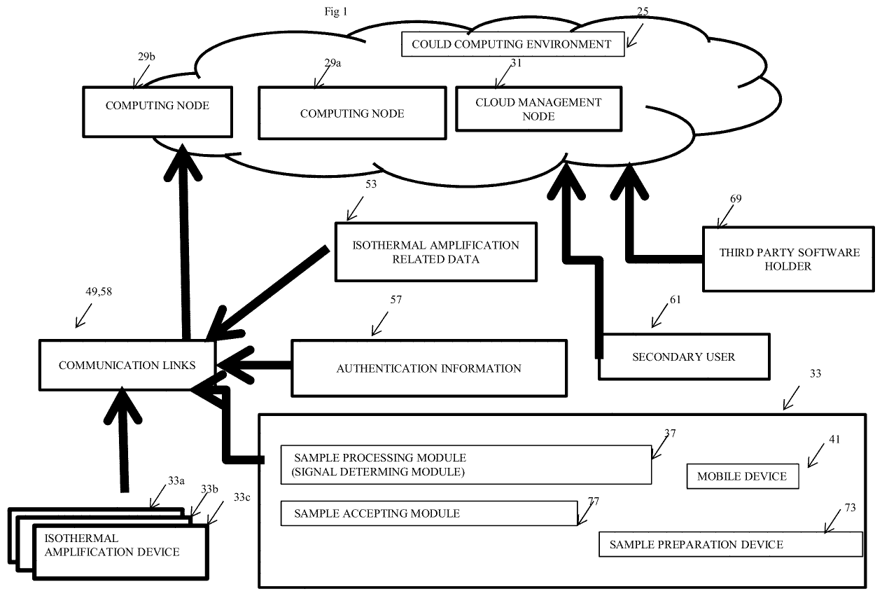

FIG. 1 is a diagrammatical overview for a system incorporating a cloud computing environment in accordance with the present disclosure. A cloud computing environment 21 for isothermal amplification information is shown in FIG. 1.

The term “cloud” or “cloud computing environment” may refer to various updating arrangement, network and infrastructure. Typically, the arrangement, network, infrastructure and the like that are related to the Internet. The cloud used herein may include any type of cloud such as client clouds, application clouds, platform clouds, infrastructure clouds, server clouds, and so on. With the arrangement, it usually can provide software as a service (SaaS, various aspects of computing platforms as a service (Paas), various network infrastructures as a service (IaaS) and so on.

By the arrangements, it should include various types of products and services. It includes but is not limited to various clouds, such as public clouds and private clouds, hybrid clouds, community clouds. Any of these cloud service may be provided by third party entities except, in certain embodiments, private clouds or hybrid clouds may only be accessed for data and service for authorized users.

A plurality of nodes 29 may be included in cloud computing environment 25. According to different customers' demand, the computing nodes 29 can be pooled together to serve multiple consumers.

The computing nodes 29 process and handle various resources including storage, processing, memory, network bandwidth, and virtual machines. The pooling nodes 29 can serve multiple customers who have different demands, via dynamically assigning and reassigning physical and virtual resources.

To communicate and manage distribution of these resources, a cloud management module 31 resides within one or more of nodes 29. The communication is through any proper arrangement or protocol. The nodes 29 may reside in different servers, which can associate with one or more providers. Different providers may be able to access or provide different resources. In one embodiment, the data storage functions could be provided by some cloud service companies, while the data analysis functions are provided by other companies. When during higher load times, some of nodes could be used as overflow nodes.

In one embodiment, the load management and cloud resource are controlled by the cloud management module or node 31. In the cloud computing environment 25, the load management may be implemented through consideration of a variety of factors, such as user access level and/or total load. For example, during peak times and average load times, the cloud resource may be accessed differently according to a user access level. The project type may be a factor, too. In one embodiment, public health emergency projects, such as a project determining the outbreak of a pandemic, will have higher priority over other types of projects. Users could choose the priority of their running projects and be charged according to the priority level.

The cloud computing environment 25 is configured to communicate with various users and devices, including mobile device users for generating nucleic acid isothermal amplification related data.

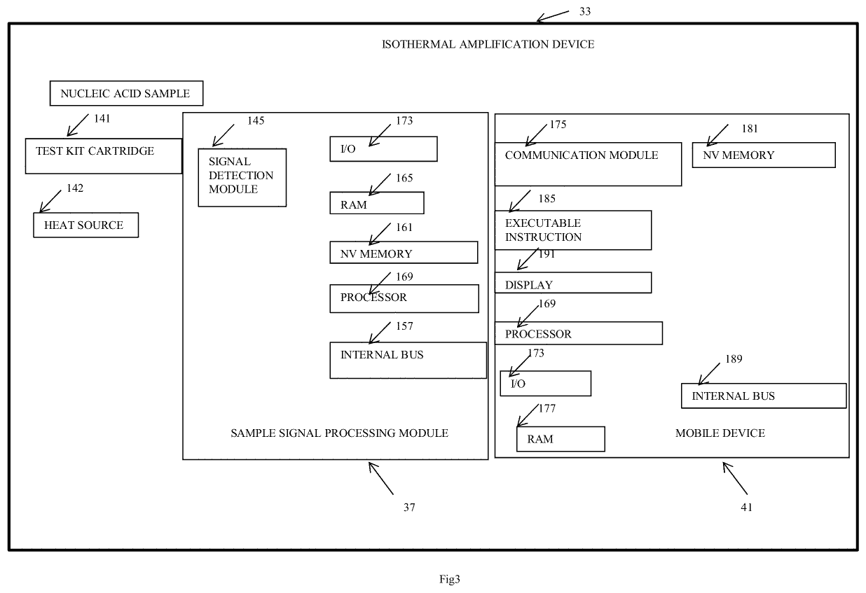

Such data may include nucleic acid amplification data generated via a signal detection module (FIG. 3, 145) of isothermal amplification device 37 that is linked to a mobile device 41, and the mobile device 41 that includes computer storage medium storing executable instructions for analyzing or communicating the isothermal amplification related data to the cloud computing environment 25. In one embodiment, the executable instructions are stored in a non-transisitor computer.

Alternatively or additionally, such data (isothermal amplification related data) may include one or more data types, such as sample preparation data (e.g., data from a bead beater) generated via a device 73 (e.g., sample preparation device such as a bead beater) and/or test subject associated data (or manifest of test subjects) provided by sample collectors/owners or collected through sensors of mobile devices.

In particular embodiments, sample preparation device 73 may include a device 77 that includes a module to accept a biological sample and generate sample preparation data (e.g., data from a bead beater or microfluidic device set) and is linked to a mobile device 41 that includes non-transitory computer storage medium storing executable instructions for analyzing or communicating the sample preparation data to the cloud computing environment 25.

In certain embodiments, the devices 33 (e.g. isothermal amplification device) and 73 may be incorporated into a single device. The devices 33, 73 are configured to communicate with the cloud computing environment 25 via a suitable communications link 49, 85. The communication with the cloud computing environment 25 may include communication via a wireless LAN (WLAN), wireless wide area network (WWAN) and/or a public network (e.g., the Internet) via the communications link 49, 58. In certain embodiments, local area network (LAN) and wide area network WAN may be used.

In particular, the communications link 49, 58 send one or more types of data (e.g. information of collectors, manifest of test subjects, environment, sample preparation related data, isothermal amplification related data) 53 and authentication information 57, to the cloud computing environment 25. The authentication information may be used to confirm that the device 33, 73 or a user device is a client of the cloud computing environment 25.

Herein the cloud computing environment 25 may serve multiple users or clients with associated devices, e.g., devices 33a, 33b, 33c, 73a, 73b, and 73c.

Further, the cloud computing environment 25 may also be accessed by other types of clients, such as secondary users 61 or third-party software holders 69. Accordingly, the cloud computing environment 25 may provide different types of services depending on the access level of the particular client. A client who carries out isothermal amplification of nucleic acid may have access to storage and data analysis services, while a secondary user 61 may only have access to the shared or public isothermal amplification related data. Third-party software holders 69 may negotiate with the clients or owners of isothermal amplification related data to determine appropriate access privileges. For instance, open source software may be offered for free or on limited license basis, while other types of software may be offered according to various fee or subscription bases.

In certain embodiments, a supplier of isothermal amplification devices or information products may support the cloud computing environment, and customers of the supplier may be given access to the cloud computing environment. For example, a purchase of a generic isothermal amplification kit/cartridge/device or service from the supplier may allow a user to access sample preparation protocols and/or software and/or isothermal amplification related data and/or receive services from the cloud computing environment. The supplier may include generic cartridge or kit providers, software developers, service providers and data brokers.

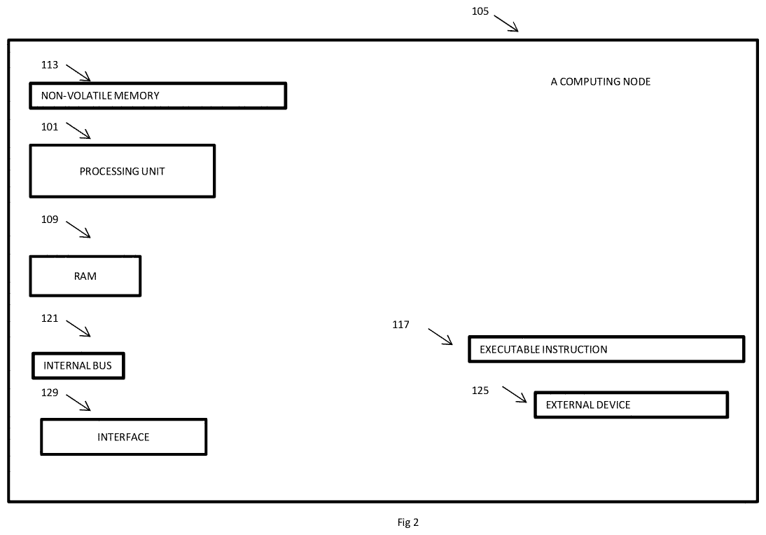

FIG. 2 is a diagrammatical overview of an individual node 29 of the cloud computing environment of the type discussed with reference to FIG. 1

FIG. 2 is a diagrammatical overview of an implementation of an individual node 29 of the cloud computing environment 25. The node 29 may be implemented as one or more of following systems or devices including a personal computer system, server computer system, thin client, thick client, hand-held or laptop device, multiprocessor system, microprocessor-based system, set top box, programmable consumer electronic, network PC, minicomputer system, mainframe computer system, and distributed cloud computing environments 25 and the like.

The node 29 may include one or more processors or processing units 101 and a memory architecture 105. The memory architecture may include RAM 109, non-volatile memory 113, removable/non-removable, volatile/non-volatile computer system storage media, one or more readers for reading from and writing to a non-removable, non-volatile magnetic media, such as a hard drive, a magnetic disk drive for reading from and writing to a removable, non-volatile magnetic disk (e.g., a “floppy disk”), and/or an optical disk drive for reading from or writing to a removable, non-volatile optical disk such as a CD-ROM, DVD-ROM. The node 29 may also include a variety of computer system readable media that may be any available media and is accessible by the cloud computing environment, such as volatile and non-volatile media, and removable and non-removable media.

The memory architecture 105 may include at least one program product with a set (e.g., at least one) of program modules. The modules are implemented as executable instructions configured to carry out the functions of the present techniques. The not limited examples of executable instructions 117 may include an operating system, one or more application programs, other program modules, and program data. Program modules may include routines, programs, objects, components, logic, data structures, and so on, that performs particular tasks or implement particular abstract data types. Program modules may carry out the functions and/or methodologies of the techniques as described herein including, but not limited to, biological source identification, disease spread model prediction (i.e. SYSTEM AND METHOD FOR TRACKING AND CONTROLLING INFECTIONS, U.S. Pat. No. 8,046,172), forensic identification analysis, genealogy analysis, pathogen identification analysis, inherited diseases, drug response prediction, reporting and advising.

The components of the node 29 may be coupled by an internal bus 121 that may be implemented as one or more of any of various types of bus structures, including a memory bus or memory controller, a peripheral bus, an accelerated graphics port, and a processor or local bus using any of a variety of bus architectures. The not limited examples of architecture include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

The node 29 may also communicate with one or more external devices such as a keyboard, a pointing device, a display or microphone 125, etc.; that allow a user to interact with the cloud computing environment 25; and/or any devices (e.g., network card, modem, etc.) that allow node 29 to communicate with one or more other computing devices. Such communication can occur via I/O interfaces 129. Additionally, the nodes 29 of the cloud computing environment 25 may communicate with one or more networks such as a local area network (LAN), a general wide area network (WAN), and/or a public network (e.g., the Internet) via a suitable network adapter. In some embodiments, the networks include wireless network such as wireless wide area network (WWAN) and wireless LAN (WLAN).

FIG. 3 is a diagrammatical overview of a sample processing module (isothermal amplification cartridge) 37 with mobile device 41 that may be used in conjunction with the cloud computing environment of the type discussed with reference to FIG. 1.

FIG. 3 is a schematic diagram of the sample amplification module 37 with mobile device 41 that may be used in conjunction with the cloud computing environment 25. In the depicted embodiment, the isothermal amplification device 33 includes a separate sample signal processing module 37 and an associated mobile device 41. However, as noted, device 41 and device 37 may be implemented as a single device. Further, the associated mobile device 41 may be local to or networked with the sample signal processing module 37. In other embodiments, the mobile device 41 may include a cloud computing environment access device that is remote from the sample signal processing module 37. That is, the mobile device 41 may be capable of communicating with the sample signal module 37 through the cloud computing environment 25. In the depicted embodiment, the nucleic acid sample may be loaded into the sample signal processing module 37, or a test kit cartridge 141 that is imaged to generate isothermal amplification data or determine isothermal amplification signals. For example, reagents that interact with the biological sample absorb or emit at particular wavelengths in response to an excitation beam generated by a signal detection module 145 and thereby return radiation for imaging. For instance, the fluorescent components may be generated by fluorescently dye molecules chelating into nucleic acids that are amplified by isothermal amplification techniques. As will be appreciated by those skilled in the art, the wavelength at which the dyes of the sample are excited and the wavelength at which they emit fluoresce will depend upon the absorption and emission spectra of the specific dyes. Such returned radiation may propagate back through the directing optics in 145. This returned radiation may generally be directed toward detection optics of the imaging module 145 or mobile device 41. Or a colorimetric method is used to determine the signal of nucleic acid amplification. The image of reaction is acquired by mobile device 41 or imaging module 145 and the color of reaction image is used to determine if the presence of any target sequence.

The optical detector of imaging module may be based upon any suitable technology, and may be, for example, a charged coupled device (CCD) sensor that generates pixilated image data based upon photons of excited or emission beam impacting locations in the device. It will be understood that any other detectors may also be used such as time delay integration (TDI) operation, an avalanche photodiode (APD) detector, a complementary metal oxide semiconductor (CMOS) detector or any other suitable detector. The imaging module 145 may be under processor control, e.g., via a processor 169, and the sample signal processing module 37 may also include I/O controls 173, an internal bus 157, non-volatile memory 161, RAM 165 and any other memory structure such that the memory is capable of storing executable instructions, and other suitable hardware components that may be similar to those described with regard to FIG. 2.

Further, the associated mobile device 41 may also include a processor 169, I/O controls 173, a communications module 175, and a memory architecture including RAM 177 and non-volatile memory 181, such that the memory architecture is capable of storing executable instructions 185. The hardware components may be linked by an internal bus 189, which may also link to the display 191. In some embodiments in which the sample processing module is implemented as an all-in-one device, certain redundant hardware elements may be eliminated. Or certain hardware elements are implemented in the mobile device instead of the sample processing module.

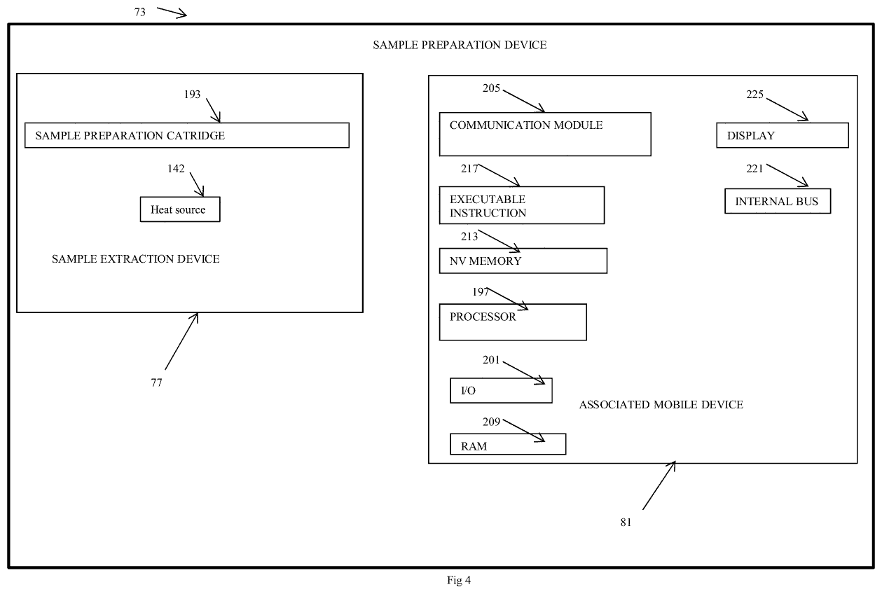

Further, a primary user (or secondary user) may also interact with the cloud computing environment 25 through any appropriate access device, such as a general purpose computer or mobile device that includes components similar to those described with regard to the mobile device 41. That is, once the isothermal amplification information (e.g. isothermal amplification related data) has been communicated to the cloud computing environment 25, further interaction with and access to the isothermal amplification information may not necessarily be coupled to the mobile device 41 (and mobile device 81 of FIG. 4). Such embodiments may be beneficial in embodiments in which the owner of the biological samples data has contracted for collecting, e.g., to crowd-source. In certain embodiments, the isothermal amplification information may be accessed through security parameters, such as a password-protected client account in the cloud computing environment 25 or association with a particular institution or IP address. The isothermal amplification information may be accessed by downloading one or more files from the cloud computing environment 25 or by logging into a web-based interface or software program that provides a graphical user display in which the isothermal amplification information is depicted as text, images and/or hyperlinks. In such an embodiment, the isothermal amplification information may be provided to the primary or secondary user in the form of data packets transmitted via a communications link or network.

The cloud computing environment 25 may execute user interaction software (e.g., via a web-based interface or application platform) that provides a graphical user interface for users and that facilitates access to isothermal amplification information, a community or group of researchers, data analysis programs, available third party software, and user selections for instrument settings, interpretations, experts. For example, in particular embodiments, settings for sample signal processing module 37 coupled with a mobile device 41(/81) may be set via the cloud computing environment 25. Accordingly, the cloud computing environment 25 and an individual mobile device 41(/81) may be capable of two-way communication. Such an embodiment may be particularly useful for controlling parameters of a remote isothermal amplification in a point-of-collection-manner.

In certain embodiment, the mobile device 41(/81) may be linked according to nanopore sequencing techniques. Some embodiments can use nanopore sequencing, whereby target nucleic acid may be unstranded or fragmented, passing through a nanopore. As the target nucleic acids or nucleotides pass through the nanopore, each oligonucleotide can be identified and quantified by measuring fluctuations in the electrical conductance of the pore (U.S. Pat. No. 7,001,792; Soni & Meller, Clin. Chem. 53, 1996-2001 (2007); Healy, Nanomed. 2, 459-481, 2007; and Cockroft, et al. J. Am. Chem. Soc. 130, 818-820, 2008.) However, only the numbers of target sequence reads are used to determine amplification of target sequence, which is based on usage of specific primers of isothermal amplification instead of sequence information. The sequencing reads are not used to reconstruct RNA or DNA sequence of a sample. Therefore, a portable next generation sequencer only serves as a signal detection module (e.g. nanopore sequencer).

FIG. 4 is a diagrammatical overview of a sample preparation device that may be used in conjunction with the cloud computing environment of the type discussed with reference to FIG. 1.

FIG. 4 is a schematic diagram of the sample preparation device 73 that may be used in conjunction with the cloud computing environment 25. (The sample preparation device 73 may be implemented according to customized user derived protocols in an automated manner.) In particular embodiments, the sample preparation device 73 may be a bead-beating device (e.g., Miniaturized bead-beating device to automate full DNA sample preparation processes for Gram-positive bacteria, Issue 21, 2011, Lab on a Chip).

In the depicted embodiment, the sample preparation device 73 includes a separate sample extraction device 77 and/or an associated mobile device 81. However, these may be implemented as a single device. Further, the associated mobile device 81 may be local to or networked with the sample extraction device 77. Furthermore, sample extraction device 77 may be implemented as a single device with isothermal amplification cartridge in device 33, and mobile device 81 may be implemented as mobile device 41.

In other embodiments, the mobile device 81 may access cloud computing environment and is remote from the sample extraction device 77. (That is, through the cloud computing environment 25, the mobile device 81 may be capable of communicating with the sample extraction device 77.) In the depicted embodiment, the biological sample may be loaded into the device 77 via a sample preparation cartridge 193. The sample preparation cartridge 193 can be utilized to convert biological samples into nucleic acid for use in isothermal amplification.

A sample preparation cartridge 193 may be a specific cartridge that is configured for use with a particular protocol or, alternatively, it may be a generic cartridge capable of being used for various protocols. For example, a specific cartridge 193 may include specific compartments and connections required for and dedicated to a specific application (e.g., isothermal amplification for RNA sample preparation). In contrast, a generic cartridge may include compartments, channels or other fluidic features that are greater in number or more variable in configuration than necessary for any single specific application of the cartridge. The use of the generic cartridge 193 allows a user to utilize a customized protocol for use with the cartridge 193 to address the specific need or application of the user. In addition, the use of the generic cartridge 193 may encourage users to utilize automated sample preparation which may result in a cost savings in reagents, while providing higher precision and reproducibility in preparing samples (e.g., DNA extraction) for isothermal amplification.

A sample preparation cartridge 193, whether specific or generic in configuration, need not include any reagents. Rather the cartridge can be supplied to a user empty and the user can subsequently load the cartridge with desired isothermal amplification sample preparation kits, reagents or fluidic components. In particular embodiments, the generic cartridge 193 may be designed for use with a microfluidics based system.

Exemplary devices and procedures for microfluidics are set forth for example in Tabeling, P. (2005). Introduction to Microfluidics., Shkolnikov, V (2019). Principles of Microfluidics., each of which is incorporated herein by reference in its entirety. Microfluidic devices are particularly suitable for a generic cartridge because the channels of microfluidic device can be designed in different ways to carry out different sample preparation protocols.

Further, the associated mobile device 81 may also include a processor 197, I/O controls 201, a communications module 205, and a memory architecture including RAM 209 and non-volatile memory 213, such that the memory architecture is capable of storing executable instructions 217 (e.g. the mobile application of the present invention). The hardware components may be linked by an internal bus 221, which may also link to the display 225. In some embodiments, the sample preparation 73 device is implemented as an all-in-one device, certain redundant hardware elements may be eliminated.

Further, a primary user (or secondary user) may also interact with the cloud computing environment 25 through any appropriate access device, such as a general purpose computer or mobile device that includes components similar to those described with regard to the mobile device 81. That is, once the isothermal amplification information has been communicated to the cloud computing environment 25, further interaction with and access to the isothermal amplification information (that includes sample preparation related data) may not necessarily be coupled to the sample preparation device 73 or isothermal amplification device 33. Such embodiments may be beneficial in embodiments in which the owner of the biological sample and/or owner of sample preparation information has contracted for sample preparation, e.g., to a crowd-source. In such embodiments, the primary user may be the owner while the crowd source associated with the sample preparation device 73 or sample extraction device 77 is at most a secondary user after the isothermal amplification information has been communicated to the cloud computing environment 25.

In certain embodiments, the sample preparation data may be accessed through security parameters such as a password-protected client account in the cloud computing environment 25 or association with a particular institution or IP address. The sample preparation information may be accessed by downloading one or more files from the cloud computing environment 25 or by logging into a web-based interface or software program that provides a graphical user display in which the sample preparation information is depicted as text, images, and/or hyperlinks and/or voice. In such an embodiment, the sample preparation information may be offered to the primary or secondary user in the form of data packets transmitted via a communications link or network.

The cloud computing environment 25 may execute user interaction software (e.g., via a web-based interface or application platform) that provides a graphical user interface for users and that facilitates access to sample preparation/isothermal amplification information, a community or group of researchers, data analysis programs, available third party software, advices or solution from experts, analysis results of isothermal amplification information and user selections for load balancing and instrument settings.

For example, in particular embodiments, the sample preparation device 73 may be set via the cloud computing environment 25. Accordingly, the cloud computing environment 25 and an individual sample preparation device 73 may be capable of two-way communication. Such an embodiment may be particularly useful for controlling parameters of a remote sample preparation run.

Furthermore, the sample preparation device 73 and the isothermal amplification device 33 may be integrated as one device. In the integrated device, redundant hardware elements may be eliminated. For example, the mobile device 41 and 81 may be the same device for the integration of sample preparation and isothermal amplification device.

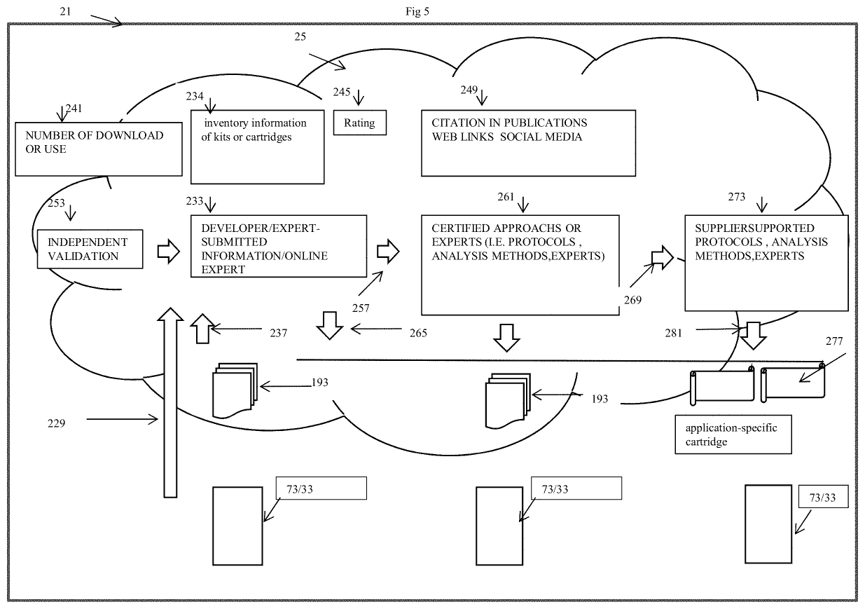

FIG. 5 is a schematic overview of a cloud-based computing environment that enables sharing of isothermal information (e.g., sample preparation protocols/isothermal amplification related data, analysis, advices and solutions of experts) and popularity monitoring.

As provided herein, the system 21 facilitates the sharing of sample preparation protocols, isothermal amplification related data, analysis, advices and solutions of experts and the monitoring of the popularity of these protocols/isothermal amplification related data, analysis, software, advices and solutions from expert via the cloud computing environment 25. To that end, FIG. 5 is a schematic diagram of an exemplary system for sharing and monitoring the popularity of various isothermal amplification information, such as isothermal amplification related data, analysis and advices and solutions from experts. The depicted cloud computing environment 25 is as described above. In certain embodiments, the cloud computing environment 25 may be supported by a supplier (e.g., manufacturer or provider) of the generic cartridges 193 or kits or software and service provider (e.g., application developers) or a product as described above for use with sample preparation devices 73 and isothermal amplification device 33.

A developer (e.g., submitter/consumer/user) uploads a customized isothermal amplification information (including optimized sample preparation protocols, primer sets, the isothermal amplification data, analysis software or advices or solutions) for use with the generic cartridges 193 or kits (i.e. isothermal amplification cartridge, sample preparation cartridge and isothermal amplification kit) or for use of with software and expert's service, to the cloud computing environment 25 as indicated by arrow 229. In one embodiment, the upload of information (optimized sample preparation protocols, primer sets, the isothermal amplification data, analysis software or advices or solutions) may be free to encourage sharing.

The protocol is used to direct the workflow of sample preparation processes. The protocols can include recipes of reagents, sequence of primer sets and procedures to drive instruments and devices (73 and 33) for processing the samples in order to generate nucleic acid amplification signal. For example, the procedures and steps may include mixing, incubation, and splitting of the samples and/or reagents; and the reagents could include combination of biological reagents such as polymerase, dyes, oligonucleotide, nucleic acids, primer sets. In addition, the protocol may specify a pre-determined amount of time and/or a temperature for each step. For instance, the protocol can provide the details for directing movement, splitting and mixing of droplets in a microfluidic device. (e.g. extraction of sample nucleic acid). In certain embodiments, the developer may also upload a corresponding analysis method for use with the uploaded protocols. The analysis method is used to interpret or analyze isothermal amplification related data. For example, the analysis method may quantify the amount of nucleic acid in the samples during the isothermal amplification reaction or determine the genotype of samples based on the primer set information and amplification signals. Further, based on the analyzed or interpreted results, experts could provide their advices or solutions. The advice and solution, for example, may include analysis report, comments on test subjects, diagnosis results, treatment for particular pathological conditions or processing methods.

The cloud computing environment 25 (e.g., memory) stores a number of developer-submitted protocols/analysis methods/advices and solutions from experts 233 for access by users (e.g., requesters/customers). These optimized protocols/analysis methods/advices and solutions 233 may encourage users to use them because the information is available in the manner of point-of-collection. Further, the users do not need waste time and resources developing all of the steps for a particular application. Users may be granted access to the cloud computing environment 25 and the protocols/analysis methods/advices and solutions from experts 233 via paying a fee to the supplier for purchasing a product. The products include but are not limited to produce, generic cartridges 193, data, software, advices from experts.

In certain embodiments, access to the protocols/analysis methods/advices and solutions from experts 233 may be limited to those users who purchase the generic cartridges or kits 193. Users with access to the protocols may request and download (e.g., directly to the sample preparation instrument 73 or isothermal amplification device 33) a particular protocol or analysis software or request advices and solutions from experts 233 for use with the generic cartridges 193 as represented by arrow 237. In some embodiments, inventory information of kits or cartridges may be submitted by users or estimated by the cloud. The inventory information of kits or cartridges can be presented by 234 as well.

The cloud computing environment 25 can monitor the usage of each type of information such as the data, protocols, analysis methods, advices and solutions (e.g. developer submitted, certified, supplier-supported).

For example, the cloud computing environment 25 monitors the number of requests or downloads 241 for each primer sets or experts' services to evaluate more specific causes for increased use of primer sets or experts' advices and solutions (e.g. an outbreak of a particular pathogen that is detectable via monitoring the usage of particular primer sets or identification of pathogens).

In some embodiments, from the service of experts' advices and solutions, the status of test subject may be properly monitored (e.g. experts can determine if a drug has significant side effect by determining certain gene expression levels or microbial contamination from the environments)

In certain embodiments, the cloud computing environment 25 monitors the number and feedback of uses for various type of information (i.e. protocols, primer sets, analysis methods, experts' advices and solution). Based on the information, suppliers and providers may adjust their products and service. In addition, the cloud computing environment 25 receives and stores ratings 245 from users of the information 233.

Further, the cloud computing environment 25 may monitor publications, forums, social media for citations and/or uses of the developer-submitted protocols and analysis methods or experts provided advices and solutions 233 (in publications, web links and social media as represented by reference numeral 249).

In addition, or alternatively, the cloud computing environment 25 may receive the various types of (i.e. publication, web links, social media) citation information from the developer and/or supplier. In either event the citations and/or relevant information from the citations can be made available to individuals or devices that access the cloud. In particular embodiments, the availability of citation information on the cloud computing environment enables users to access the developer-submitted protocols and analysis methods or experts provided advices and solutions 233 directly without looking through multiple information resources to find solutions, advices, primer sets, protocol and kits to address the particular needs. For certain protocols, the supplier of generic cartridge or information providers may perform independent validation, as represented by reference numeral 253, of the submitted information 233 (solutions, advices, primer sets, protocol and kits).

Based on a combination of the ratings, citations in publications, forums, social media 249, and/or supplier validation, particular user or developer-submitted information 233 (Data, solutions, advices, primer sets, protocol and kits) may be conferred with a certified status as represented by arrow 257 to become certified approaches or expert 261. The certified status of the approaches or experts 261 may encourage more users to use a particular approach or expert as represented by arrows 265.

In turn, more users may be encouraged to obtain cartridges (e.g. generic cartridges 37 and/or 193 from the supplier) or information (e.g. data, software or service from information providers). The certified status may be determined by the cartridge supplier or information provider based on information obtained from the cloud computing environment 25. Alternatively, the cloud computing environment 25 (e.g., processor) may determine whether to confer the certified status based on executable instructions or criteria provided to the cloud computing environment 25.

The supplier via the cloud computing environment 25 monitors the popularity (e.g., number and frequency of requests and/or uses 241) for all types of isothermal amplification information that is related to test subjects. A supplier may identify a niche application with market potential from among the certified protocols, software, advice or solution 261 (e.g., sample preparation recipes, corresponding analysis methods, experts' advices or solutions). Upon identifying such a niche application, the supplier via the cloud computing environment 25 may confer a supplier-supported status as represented by arrow 269 on the certified approach 261 to provide supplier-supported protocols, analysis software, advice and solution 273. In addition, the supplier may generate, design, or commercialize an application-specific cartridge 277 (e.g., pre-filled with reagents) based on the supplier-supported protocol 273. The user of the application-specific cartridge 277 may download or retrieve the supplier-supported protocol 273 for use with the cartridge 277 as represented by arrows 281. The supplier-supported protocols 273 may also encourage even more users to obtain application-specific cartridges 277, sample preparation instruments 73, and/or isothermal amplification device 33, and/or related consumables, e.g., from the supplier.

To encourage sharing of protocols, the supplier via the cloud computing environment may provide credit to the submitter or developer of the protocol 233 submitted to the cloud computing environment 25 for each user requests for the submitted protocol 233. This credit may be used for purchasing consumables (e.g., cartridges or fluid components), devices (e.g. sample preparation) or services (e.g. custom data analysis, medical diagnosis or alternative sample analysis) from the supplier.

As discussed above, the system 21 facilitates interactions between protocol or software developers, requesters, the supplier, and the cloud computing environment 25 in terms of sharing and monitoring the popularity of various information related to isothermal amplification of test subjects' nucleic acids including test subjects' information, protocols, sequences of primer sets, analysis software, advices from experts.

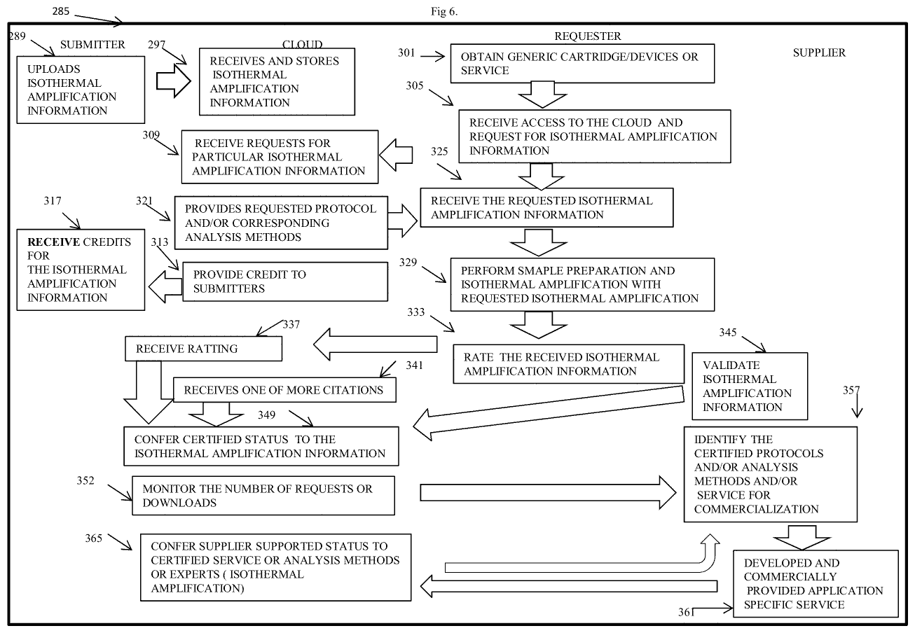

FIG. 6 is a flow diagram of a method of interaction of submitters, requesters, and a supplier with respect to the sharing and monitoring of the data, protocols, analysis methods, advices and solutions on the cloud-based computing environment of the type discussed with reference to FIGS. 1 and 5.

To that end, FIG. 6 is a flow chart of a method 285 of some exemplary interactions for sharing and monitoring the popularity of isothermal amplification information via the cloud computing environment 25.

The method 285 may include any viable subset, combination, or modification of the steps or interactions depicted.

In one embodiment, the method 285 may begin with the submitter (e.g., developer) developing and submitting the isothermal amplification information (i.e. protocol, data, analysis method, advice and solution) (block 289).

The isothermal amplification information may include data, optimized protocols, analysis software, experts' advice and solutions. For example, the developer may use the generic isothermal amplification or sample preparation device/cartridge (37/193/141/77) for the optimized protocols. It could also begin with the submitter developing software that is used to analyze the isothermal amplification related data for test subjects.

Further, it could begin with the submitter providing advices or solutions based on the isothermal amplification information.

The submitter uploads the isothermal amplification information to the supplier-supported cloud computing environment 25 (block 289), for example, without charge. The cloud computing environment 25 receives and stores the isothermal amplification information (block 297). In one embodiment, a user can submit and upload the isothermal amplification related data to the cloud. In one embodiment, submitters and requesters can be the same person. They may request the isothermal amplification related information on the cloud for analysis and/or further submit advice and solution.

The method 285 may include the requester (e.g., consumer) obtaining (e.g. by commercial purchase) the generic cartridge/device 193/37 or service from the supplier, in turn, giving the requester access to the cloud computing environment 25 (block 301). Upon receiving access to the cloud computing environment 25, the requester requests a particular protocol and/or corresponding analysis method and/or particular service from among the available protocols, corresponding analysis methods and/or experts' services (block 305). The method 285 could also include the requester purchase access to cloud 25 for the isothermal amplification related data of test subjects, as well as services from experts for specific applications.

The cloud computing environment 25 receives requests for the particular isothermal amplification information (block 309).

The cloud computing environment 25 provides credit to the submitter of the requested isothermal amplification information from the supplier (block 313), which the submitter of the requested isothermal amplification information receives (block 317) for each request and/or use of the isothermal amplification information.

The cloud computing environment 25 also provides the requested protocol and/or corresponding analysis method to the requester (block 321). Upon receiving the requested isothermal amplification information (block 325), the requester performs sample preparation and isothermal amplification with the generic cartridge 193 using the requested protocol and/or corresponding analysis method and/or experts' service or advices (block 329).

The method 285 may include the requester rating the protocol and/or corresponding analysis method and/or solution and providing the rating to the cloud computing environment (block 333). The cloud computing environment 25 receives the rating for the protocol and/or corresponding analysis method and/or services (block 337) from the requester.

Additionally, the cloud computing environment receives one or more citations from publications that cite and/or use the isothermal amplification information (block 341). The method 285 may also include the supplier performing independent validation of the submitted isothermal amplification information (block 345). Based on a combination of ratings, publication citations, and/or supplier validation of the submitted isothermal amplification information, the cloud computing environment 25 confers the certified status to the isothermal amplification information (e.g., protocol and/or corresponding analysis method and/or services) (block 349). As mentioned above, the certified status may be determined by the supplier based on information (such as citation of publication) obtained from the cloud computing environment 25. Alternatively, the cloud computing environment 25 (e.g., processor) may determine whether to offer the certified status based on executable instructions or criteria provided to the cloud computing environment 25.

The method 285 includes monitoring the number of requests or downloads of the submitted isothermal amplification information (pre- and post-certification) (block 352). In certain embodiments, the method 285 may include monitoring the number of uses of the requested or downloaded isothermal amplification. Based on the number of requests and/or the number of uses and other information (e.g., ratings, consumer demand for application, market considerations, etc.), the supplier identifies if the certified protocol and/or corresponding analysis method and/or service is commercializable (e.g., a niche application with market potential) (block 357). If the certified protocol/method/service is deemed commercializable, the supplier develops and commercially provides the application-specific service based on particular protocols and methods (block 361). In addition, the method 285 includes conferring a supplier-supported status to the certified protocol and/or analysis method and/or expert on the cloud computing environment (block 365).

As mentioned above, in certain embodiments, the cloud computing environment 25 may be used to guide an isothermal amplification workflow from beginning to end. Examples of cloud-guided isothermal amplification workflows include, but are not limited to, pathogen identification, environmental monitoring, sample preparation for diagnosis of cancer types based on patients' samples' SNP, targeted amplification, SNP for haplotyping, and low input sample preparation (e.g., forensic purposes, single cell, virus-infected tissues).

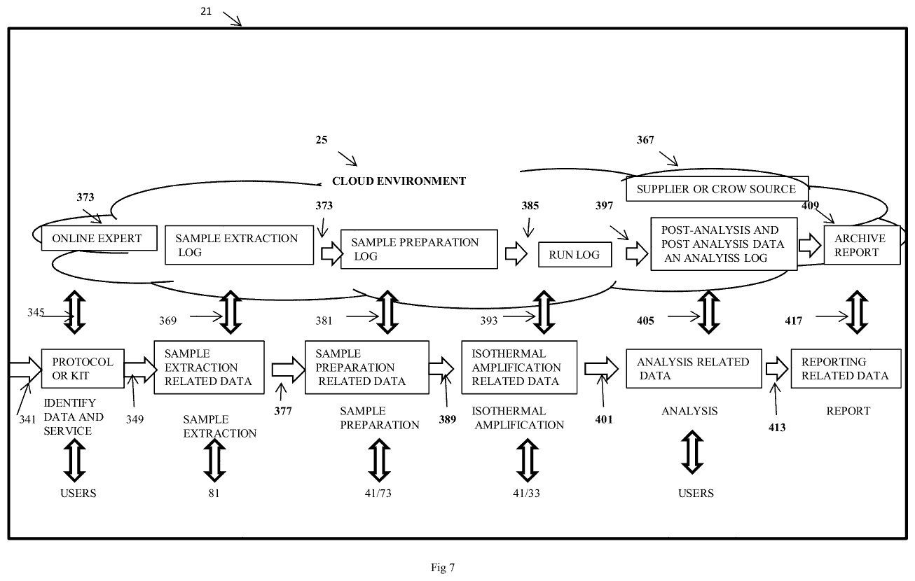

FIG. 7 is a schematic overview of a cloud-based computing environment to facilitate a cloud-guided isothermal amplification information analysis workflow

To that end, FIG. 7 is a schematic overview of the cloud-based computing environment 25 to facilitate a cloud-guided isothermal amplification data analysis workflow. In certain embodiments, the cloud computing environment 25 may be supported by a supplier (e.g., manufacturer/provider) of products and/or instruments used in the isothermal amplification analysis workflow. FIG. 7 depicts the major steps involved in a typical isothermal amplification information analysis workflow. In certain embodiments, additional steps may be included or some steps are not performed. Some of the steps (e.g., analysis and reporting steps as well as experts giving advice and solutions) may be performed from computing devices with access to the cloud computing environment 25. In general, upon gathering information (e.g., identity of test subjects or users, conditions, parameters) required for each of the steps, the information is provided to the cloud computing environment 25 via computing devices or instruments. Certain sources of these parameters or information may include information from a barcode- or RFID-tracked sample plates, sample preparation cartridges, isothermal amplification cartridges, isothermal amplification kits and other sources. In addition, various manifests and recipes (e.g., protocols) reside in the cloud computing environment 25 (e.g., memory). These procedures and recipes are provided to the instruments or mobile devices (e.g., sample preparation instrument 73, isothermal amplification device 33, etc.) to drive the specific steps (e.g., sample preparation, isothermal amplification, etc.). Once the specific tasks or steps begins, data and instrument feedback are provided to cloud computing environment 25 for further steps (logging, analysis, report generation . . . etc.). The various analysis methods, report formats and advising services also reside in the cloud computing environment 25. Also, the various sample preparation recipes (e.g., protocols), analysis methods, report formats, and services may be developed by the supplier or crowd-sourced (e.g., see FIGS. 5 and 6) as indicated by reference numeral 367. The steps of workflow in the cloud computing environment 25 parallel the steps in the laboratory. This enables the cloud computing environment to act as a workflow manager (e.g., in an application-centric fashion) to guide the physical process from start to complete.

In one embodiment the workflow may begin with a user choosing the service or data that they would like to have (as represented by arrow 341). After the selection of service or data type, the online experts may provide suggestion for the protocol or kits for the isothermal amplification (as represented by arrow 345). The user purchases the kits or cartridge from suppliers (as represented by arrow 349).

After receiving the kit and devices for isothermal amplification, the workflow starts with identification of test subject(s), order of information product or service, and sample extraction from a biological source. A sample manifest residing on the cloud computing environment 25, as well as the instruction for sample extraction (e.g., sample manifest provided by the user or another source, the instruction provided by the cloud), is provided to the user as represented by arrow 369. Upon and/or during sample extraction, a user provides sample extraction related data (sample identification, sample plate identification, plate position identification, extraction yield, other parameters, etc.) to the cloud computing environment 25 via, e.g., mobile device 81 or a computing device as represented by arrow 370. Based on the sample extraction related data and/or sample manifest, the cloud computing environment 25 (e.g., processor) generates a sample extraction log.

After sample extraction, the workflow shifts to sample preparation as indicated by arrows 373, 377. The sample preparation device 73 (via a mobile device) or the user (or third party) via a different computing device provides sample preparation related data (e.g., sample preparation recipe/protocol identification, sample preparation cartridge identification, cartridge preparation identification, sample preparation instrument identification, other parameters, etc.) to the cloud computing environment 25 as represented by arrow 381.

In turn, the cloud computing environment 25 provides a sample preparation recipe and sample preparation manifest to the sample preparation instrument 73 to drive the sample preparation as represented by arrow 381. In some embodiments, the sample preparation protocol or recipe used by the sample preparation instrument 73, via the instructions from the cloud computing environment 25, may be based on a protocol selected by a user, a protocol selected or instructed by a third party, or a protocol automatically loaded based on sample or cartridge identification.

Upon and/or during sample preparation, sample preparation data is provided as shown by arrow 381 to the cloud computing environment 25. Based on the sample extraction log, sample preparation related data, sample preparation data, sample preparation recipe, and/or sample preparation recipe, the cloud computing environment 25 (e.g., processor) generates a sample preparation log.

After sample preparation, the workflow shifts to isothermal amplification as indicated by arrows 385, 389. The isothermal amplification device 33 or the user (or third party) provides isothermal amplification related data (e.g., manifest, etc.) to the cloud computing environment 25 as represented by arrow 393. In some embodiments, the isothermal amplification protocol used by the isothermal amplification device 33, via instructions from the cloud computing environment 25, may be based on a protocol selected by a user, a protocol selected or instructed by a third party, or a protocol automatically loaded based on the isothermal amplification related data. Upon and/or after isothermal amplification, the isothermal amplification device 33 provides isothermal amplification data to the cloud computing environment 25 via mobile device 41. Based on the sample preparation log, isothermal amplification related data, the cloud computing environment 25 (e.g., processor) generates run data and a run log.

After isothermal amplification, the workflow shifts to analysis as indicated by arrows 397, 401. The isothermal amplification device 33 or the user via a different computing device provides analysis related data (e.g., post-analysis data, analysis identification, other parameters, etc.) to the cloud computing environment 25 as represented by arrow 405. In certain embodiments, the cloud computing environment 25 performs the analysis (e.g., primary, secondary, and/or tertiary analysis) using the analysis method, the run data, and/or the run log. In some embodiments, the mobile device performs some of the analysis (e.g., primary and/or secondary analysis). In other embodiments, a different computing device may perform the analysis (e.g., primary, secondary, and/or tertiary analysis). In certain embodiments, the analysis may be crowd-sourced 367. Based on the run data, run log, analysis related data, and/or analysis method, the cloud computing environment 25 (e.g., processor) generates post-analysis data and an analysis log.

After analysis, the workflow shifts to reporting as indicated by arrows 409, 413. The user via a different computing device provides reporting related data (e.g., report identification, share privileges, other parameters, etc.) to the cloud computing environment 25 as represented by arrow 417. In turn, the cloud computing environment 25 may provide a report format and/or service to the user on the computing device as represented by arrow 417. In certain embodiments, the cloud computing environment 25 performs the reporting and/or service using the post-analysis data, analysis log, report format and/or reporting related data. In other embodiments, the user may perform the reporting and/or service on a different computing device. In certain embodiments, the reporting and/or service may be crowd-sourced 367. Based on the post-analysis data, analysis log, reporting related data, report format, service, the cloud computing environment 25 (e.g., processor) generates an archived report.

As discussed above, the system 21 facilitates interaction between users (e.g., primary and secondary users), the supplier, and the cloud computing environment 25 to facilitate the analysis workflow of isothermal amplification data. In particular, the cloud computing environment 25 and information stored therein serves as a workflow manager to guide the physical process from start to end in an application-centric fashion as the samples are physically moved through the various steps of the workflow of isothermal amplification of nucleic acids.

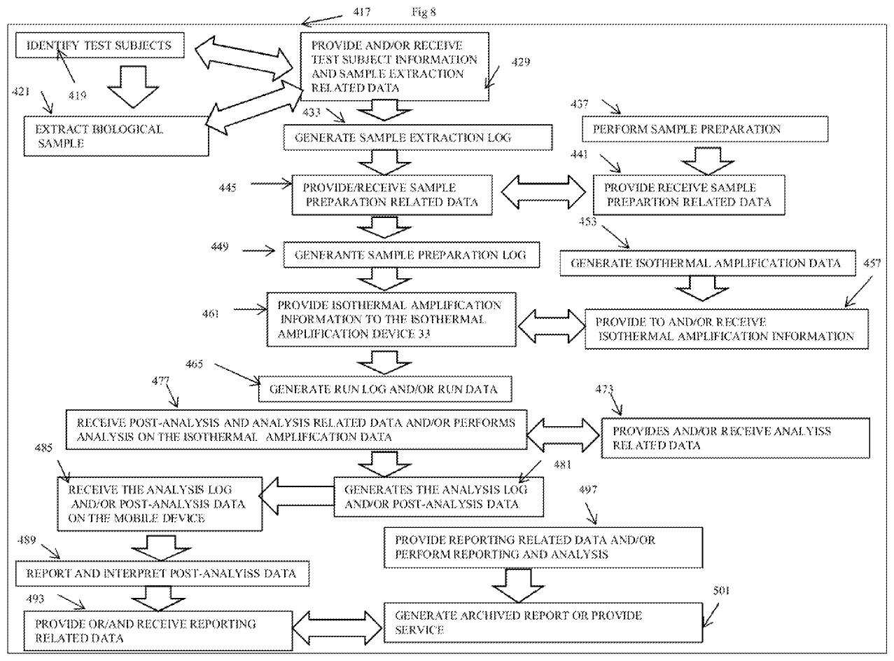

FIG. 8 is a schematic overview of a flow diagram of a method of interaction of a user and devices with the cloud-based computing environment of the type discussed with reference to FIGS. 1 and 7.

To that end, FIG. 8 is a flow chart of a method 417 of some exemplary interactions for a cloud-guided isothermal amplification analysis workflow. The method 417 may include any combination, viable set, or modification of the depicted steps or interactions. Certain steps of the method 417 may be involved with crowd-source. Certain steps of the method 417 performed by the user may be also performed by distinct users (e.g., primary and secondary users).

In one embodiment, the method 417 may begin by a user identifying the test subjects (i.e. water or food collected on site, patients' tissue, block 419) and extracting one or more biological samples from test subjects (e.g. microbial in water, block 421). The user provides to and/or receives from the cloud computing environment 25 for test subject information (e.g. sample manifest) (block 419) and/or sample extraction related data (block 421), e.g., via a computing device. For example, the user may provide test subject information, sample plate identification, plate position identification, or other parameters to the cloud computing environment 25 for storage (e.g., memory) and/or processing (e.g., processor).

In turn, the cloud computing environment 25 (e.g., server) provides and/or receives test subject information and sample extraction related data to the user (block 429). For example, the cloud computing environment 25 may provide test subject information or sample extraction log to the user. In certain embodiments, at least some of the test subject information and/or sample extraction related data may be provided to the user from the cloud computing environment 25 prior to sample extraction (block 421).

Based on the sample extraction related data received from the user and/or the test subject information from the cloud computing environment 25, the cloud computing environment 25 (e.g., processor) generates the sample extraction log (block 433).

Following sample extraction, the method 417 includes performing sample preparation on the sample preparation instrument 73 (e.g., automated sample preparation instrument) (block 437). The sample preparation instrument 73 provides to and/or receives from the cloud computing environment 25 sample preparation related data (e.g., protocols) (block 441).

In certain embodiments, the user provides and/or receives the sample preparation related data via another computing device connected to cloud. For example, the sample preparation instrument 73 may provide sample preparation protocol identification, sample preparation cartridge identification, sample preparation cartridge location identification, sample preparation instrument identification, generated sample preparation data, and other parameters to the cloud computing environment 25 for storage (e.g., memory) and/or processing (e.g., processor).

In certain embodiments, the instrument 73 provides the generated sample preparation data to the cloud computing environment 25 during and/or after the generation of the data.

In turn, the cloud computing environment 25 (e.g., server) provides sample preparation related data (e.g., protocols) to the sample preparation instrument 73 and/or user, and/or receives the sample preparation related data (block 445). For example, the cloud computing environment 25 may provide the sample extraction log, sample preparation protocols, sample preparation manifest, and/or sample preparation log to the instrument 73 and/or user.

In certain embodiments, at least some of the sample preparation related data (test subject information) may be provided to the instrument 73 prior to sample preparation (block 437). The sample preparation protocol and other information may be used to drive the sample preparation instrument 73. Based on the sample extraction log, sample preparation information and/or generated sample preparation information received from the sample preparation instrument 73 and/or cloud computing environment 25, the cloud computing environment 25 (e.g., processor) generates the sample preparation log (block 449).

Following sample preparation, the method 417 includes generating isothermal amplification data on the isothermal amplification device 33 (block 453), and isothermal amplification device 33 provides to and/or receives from the cloud computing environment 25 isothermal amplification information (e.g., protocols, signal analysis method) (block 457). In certain embodiments, the user provides and/or receives the isothermal amplification information via another computing device. For example, the isothermal amplification device 33 may provide cartridge identification, mobile device identification, generated isothermal amplification data, and other parameters to the cloud computing environment 25 for storage (e.g., memory) and/or processing (e.g., processor). In certain embodiments, the isothermal amplification device 33 provides the generated isothermal amplification data (e.g., presence of certain nucleic acid sequences, image of reactions) to the cloud computing environment 25 during and/or after the generation of the data. In turn, the cloud computing environment 25 (e.g., server) receives the isothermal amplification information from isothermal amplification device 33 and/or provides isothermal amplification information to the isothermal amplification device 33 (block 461) and/or user. For example, the cloud computing environment 25 may provide the sample preparation log, task instructions, run data and/or a run log to the isothermal amplification device 33 and/or user. In certain embodiments, at least some of the isothermal amplification information (e.g., task instructions) may be provided to the isothermal amplification device 33 prior to isothermal amplification of test subject's nucleic acid (block 453). The task instructions and other information may be used to drive the isothermal amplification device 33. Based on the sample preparation log, isothermal amplification information, and/or generated isothermal amplification information received from the isothermal amplification device 33 and/or cloud computing environment 25, the cloud computing environment 25 (e.g., processor) generates the run log and/or run data (block 465).

Following isothermal amplification, the method 417 includes analyzing the isothermal amplification data (e.g., primary and/or secondary analysis) on the isothermal amplification device 33 (block 453). The isothermal amplification device 33 provides and/or receives from the cloud computing environment 25 analysis related data (block 473). In certain embodiments, the user provides and/or receives the analysis related data via another computing device. For example, the isothermal amplification device 33 may provide analysis identification, post-analysis data, and/or other parameters to the cloud computing environment 25 for storage (e.g., memory) and/or processing (e.g., processor). In certain embodiments, the isothermal amplification device 33 provides the post-analysis data to the cloud computing environment 25 during and/or after the generation of the data. In turn, the cloud computing environment 25 (e.g., server) receives the post-analysis and analysis related data from the isothermal amplification device 33 and/or performs analysis (e.g., primary, secondary, and/or tertiary analysis) on the isothermal amplification data via at least one processor (block 477). For example, the cloud computing environment 25 may provide an analysis method to the isothermal amplification device 33 prior to analyzing the isothermal amplification data. As mentioned above, the analysis (e.g., primary, secondary, and/or tertiary analysis) of the isothermal amplification data may be crowd-sourced. Based on the run data, run log, analysis related data, and/or analysis method received from the isothermal amplification device 33 and/or cloud computing environment 25, the cloud computing environment 25 (e.g., processor) generates the analysis log and/or post-analysis data (block 481). The user receives the analysis log and/or post-analysis data on the mobile device 41 of the isothermal amplification device 33 or another computing device (block 485).

Following analysis of the isothermal amplification data, the method 417 includes the user reporting and interpreting the post-analysis data (block 489) via the mobile device 41 or another computing device. In certain embodiments, the reporting and interpretation of the post-analysis data may be crowd-sourced or from experts. The user provides to and/or receives from the cloud computing environment 25 reporting related data (block 493). For example, the user provides via a computing device a report identification, share privilege information, and/or any reported and/or interpretation data to the cloud computing environment 25 for storage (e.g., memory) and/or processing (e.g., processor). In turn, the cloud computing environment 25 (e.g., server) provides reporting related data to the user and/or performs the reporting and analysis on the post-analysis data via at least one processor (block 497) or experts. For example, the cloud computing environment 25 may provide a report format or service, and/or an archived report to the user. Based on the post-analysis data, analysis log, report format, annotation plug-in, and/or reporting related data from the user and/or cloud computing environment 25, the cloud computing environment 25 (e.g., processor) generates the archived report or provide service (block 501). In certain embodiment, the cloud service and analysis methods can be hosted by Genalyze LLC (Delaware).

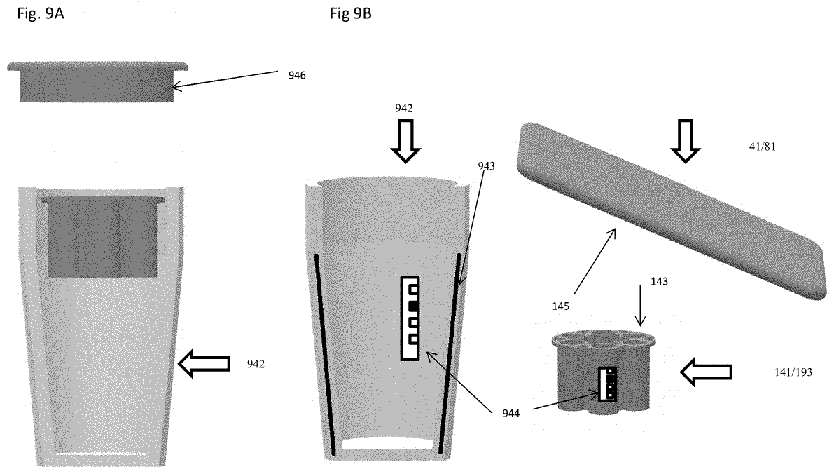

FIGS. 9A and 9B are a depiction of the cartridge for the sample processing module of the isothermal amplification device. FIG. 9A is a longitudinal cross-sectional view of a thermal container 942 with a cartridge 141/193 positioned near the top. FIG. 9B is an exploded view of the thermal container with the cartridge 141/193 removed and links to a mobile device 41/81 having installed thereon a mobile application of the present invention.

In the depicted embodiment of FIG. 9B, the thermal container 942 comprises a closed bottom end 944 and an opened top end 946 that is able to be sealed closed by a lid 946; and may further comprise: processor, memory, and a power source (e.g. battery and/or an electrical power cord). Thermal container 942 further comprises a thermal storage medium 943 inside the container (e.g. in the inner wall) and is adjacent to a heating material (e.g. water under the cartridge), and a conductive material (e.g., metal inner wall or water) places the heating material in thermal communication with a thermal storage medium 943, the thermal storage medium 943 regulates the temperature of heating material which also has thermal communication with cartridge 141/193 of a test kit or other component of the system and transmits temperature information to an associated (e.g. Bluetooth or wireless chip or USB linked) mobile device 41/81.

The thermal indicating label 944 is used to monitor the temperature of heating material or cartridge 141/193. The camera of a mobile device 41/81 can serve as a signal detection module 145. Cartridge 141/193 can hold the reaction. The isothermal amplification information (e.g. image of isothermal amplification reaction) is acquired through the camera, and is further processed by mobile device 41/81 and uploaded to the cloud.

Cartridge 141 further comprises a plurality of wells or reaction chambers 143, and each well is able to hold a nucleic acid prepared from a sample preparation device. For example, each well 143 can hold a unique patient sample for a isothermal amplification reaction (LAMP), and the other reactants required (e.g. primers, DNA polymerase, etc.).

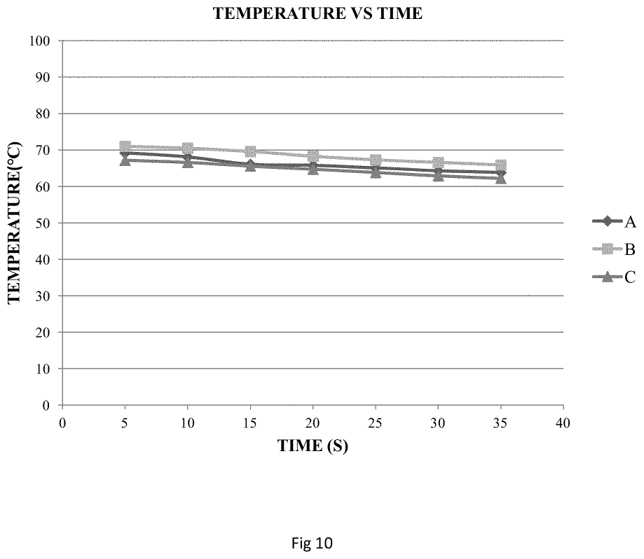

FIG. 10 is the chart of temperature versus time after the isothermal amplification device is heated up or activated by boiled water. The A, B, C stands for the temperature measurements at a series of times at 5, 10, 15, 20, 25, 30, 35 minute after boiled water poured into the isothermal amplification device

### EXEMPLIFICATIONS

### Example 1

For example, saliva collected from a test subject of SARS2-COVID may be dropped into a sample preparation device—a cartridge with reagents. After mixing, the saliva and reagent mixture is dispensed into the isothermal amplification device and it starts the isothermal amplification process. The result of the isothermal amplification and other related information for the test subject can be sent to the cloud via the test subject's mobile device. If the test subject is infected, the information will report to local health authority and the test subject's location of history may be used to predict the spread of SARS2-COVID and inform any contact in proximity of the test subject.

### Example 2

Consider a user would like to know if she/he has Usher syndrome. She/he may order a service, and the service supplier would deliver an isothermal amplification kit comprising a sample preparation and isothermal amplification cartridge with a pre-designed primer set. The user may follow the instructions and install the corresponding mobile application of the present invention (e.g. a computer program product) on her/his mobile device. The application would instruct the user on how to carry out all necessary procedures to complete isothermal amplification with his/her body fluid. The pre-primer set offered by the supplier maps to the PCDH15 gene (deletion allele of PCDH15 gene, Variation ID: 633454 on NCBI ClinVar). The deletion allele is associated with deficiency. By following the instruction shown on the mobile device, the user may extract his/her own genomic DNA from his/her body fluid and perform isothermal amplification. The mobile device associates with isothermal amplification kit and cartridge may determine which allele type she/he carries. Once isothermal amplification data is available on the site of collection and if she/he does have the deficiency allele, the mobile device may send this information to a remote cloud; and lookup on a database for a genetic counselors list in her/his residential area. She/he may contact the online/local genetic counselors for the immediate guidance.

### Example 3

A user would like to know her/his ancestry. She/he could order a kit containing the primer set for SNP listed in (e.g., Selecting SNPs to Identify Ancestry, Ann Hum Genet. 75(4): 539-553, 2011.) and an isothermal amplification kit with a cartridge from a supplier, and she/he would install the corresponding mobile application of the present invention on her/his mobile device. She/he could perform isothermal amplification techniques. Once the SNP information is available, the mobile device could analyze and determine her/his ancestry. The method described in (Selecting SNPs to Identify Ancestry, Ann Hum Genet. 75(4): 539-553, 2011.) or other more favorable methods on the cloud can be chosen to perform the analysis. The user can decide if she/he would like to associate with other users on the cloud or other social media based on their SNP profile or analysis results.

### Example 4

A biological control consulting company would like to know if the tomato its clients planted got infected with certain pathogens such as Pythium species bacteria. A biological control consulting company could use the primer sets listed in (Use of LAMP Detection to Identify Potential Contamination Sources of Plant-Pathogenic Pythium Species in Hydroponic Culture Systems of Tomato and Eustoma, Plant Dis. 102(7):1357-1364, 2018) or others that are shared on the cloud. The company can order isothermal amplification kits with cartridges for its clients. Its clients install corresponding application on her/his mobile devices. She/he could perform isothermal amplification techniques by following the interactive instruction on the mobile device. Once the isothermal amplification data is available, the mobile device could send the data to cloud. All sample records can associate with the locations where the soil samples are taken as well as the photos of plants and sampling time. Biological control specialists from the companies may analyze the isothermal amplification data and contact the clients and provide advice for treatment. Alternatively, biological control companies can access the isothermal amplification data or report from their clients and may bid or provide quotes for the treatment project.

### Example 5

A user would like to know if the tomato he/she planted got infected with certain pathogens, such as Pythium species bacteria. She/he could order a kit containing the pre-designed primer set listed in (Use of LAMP Detection to Identify Potential Contamination Sources of Plant-Pathogenic Pythium Species in Hydroponic Culture Systems of Tomato and Eustoma, Plant Dis. 102(7):1357-1364, 2018) and an isothermal amplification kit with cartridges, and then install the corresponding mobile application of the present invention on her/his mobile device. She/he could perform isothermal amplification techniques. Once the isothermal amplification is available, the mobile device could send the data to the cloud. If it is infected by the pathogens in the panel, an online/local biological control specialist may contact him/her and provide advice for treatment.

### Example 6

A user would like to identify a victim's corpse in a catastrophe. She/he could use the isothermal amplification kit and cartridge with a primer set listed in (Forensic Science International 149, 279-286, 2005), and then install the corresponding mobile application of the present invention on her/his mobile device. She/he could perform isothermal amplification techniques with DNA extracted and prepared by a sample preparation kit from the victim. Once the isothermal amplification data is available, the mobile device could send the data to cloud. A method described in (Allele frequencies for 70 autosomal SNP loci with U.S. Caucasian, African-American, and Hispanic samples, Forensic Science International 149, 279-286, 2005) or other more favorable method can be used to identify the victim.

### Example 7

A user would like to identify certain bacterial species with a lower population from a subject in a resource limited location. She/he could use the isothermal amplification kit and nanopore sequencer with some primer sets, and then install the corresponding mobile application of the present invention on her/his mobile device. She/he could perform isothermal amplification techniques with nucleic acid extracted and prepared by sample preparation kit from the subject. She/he could load the amplified nucleic acid into a nanopore sequencer for sequencing and determine the amount of amplified nucleic acid, and the mobile device could send the data to cloud. An epidemiologist could determine a spread model of the disease which is caused by said bacterial species.

### Example 8

A buyer would like to know if the fruit purchased on the supermarket was originally from a particular supplier. He could order an isothermal amplification kit and a pre-design primer set. After installing the mobile application of the present invention on his/her cell phone, he/she could follow the instructions displayed on his/her mobile device to finish sample preparation and processing procedure. The cell phone uploads the isothermal amplification related data. The program on the cloud matches the entries on the database and identifies the source of the fruit, preparing the report and delivering the analysis results to the users.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings. The transitional term “comprising”, which is synonymous with “including,” “containing,” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Or, the technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed.

As used herein, the term “about” refers to plus or minus 10 percent of the stated amount.

As used herein, the term “substantially” refers to what one of ordinary skill in the art would recognize a significantly similar.

1-20. (canceled)

21. An isothermal amplification device linked to a mobile device, said isothermal amplification device comprising:
a) a component comprising a heat source for conducting an isothermal amplification reaction, said heat source maintains said component within a specific range of temperature from 25 deg. C. to 80 deg. C., wherein said specific range of temperature is suitable for said isothermal amplification reaction; and
b) a test kit comprising a cartridge component positioned next to said heat source, wherein said cartridge component is suitable for conducting said isothermal amplification reaction within and generate an isothermal amplification information;
wherein said mobile device receives said isothermal amplification information, and processes and wirelessly transmits said isothermal amplification information to a cloud computing environment;
wherein said cloud computing environment is configured to communicate with various users and devices, including mobile device users of said isothermal amplification information, wherein said isothermal amplification reaction is conducted for a period of time from about 5 minutes to about 120 minutes, and in an ambient temperature ranging from 0 deg. to 50 deg. C.

22. The isothermal amplification device of claim 21, wherein said heat source comprises a heating element with a preset temperature for said isothermal amplification reaction.

23. The isothermal amplification device of claim 21, wherein said heat source further comprises a phase change material which serves as a thermal storage medium.

24. The isothermal amplification device of claim 21, wherein said heat source is able to be heated up or activated by: a microwave oven, an oven, a hot plate, or by adding a high temperature fluid.

25. The isothermal amplification device of claim 21, wherein said heat source or at least one component of said test kit is placed adjacent to a temperature indicating label whereby the temperature of said heat source or at least one component of said test kit is monitored by a color change of said temperature indicating label.

26. The isothermal amplification device of claim 25, wherein said test kit further comprises a negative control sample or a positive control sample.

27. The isothermal amplification device of claim 21, wherein said test kit further comprises one or more of: DNA polymerase, reverse transcriptase, nucleotides, reaction buffers, and nucleic acid primers.

28. The isothermal amplification device of claim 21, wherein said cartridge component comprises a plurality of wells, and each well is able to hold a nucleic acid prepared from a sample preparation device comprising a container storing reagents for cell lysate of a sample.

29. The isothermal amplification device of claim 28, wherein said sample preparation device further comprises a bead beater capable of processing one or more samples.

30. A method for processing and sharing isothermal amplification information, the method comprising the steps of:
a) providing a networked computerized system comprising:
i) an isothermal amplification device for amplifying one or more sample nucleic acid, said isothermal amplification device comprising: a heat source, said heat source is able to maintain a specific range of temperature suitable for an isothermal nucleic acid amplification reaction; and a test kit comprising a cartridge component positioned next to said heat source, said cartridge component is suitable for conducting said isothermal nucleic acid amplification reaction within and generate an isothermal amplification information;
ii) a mobile device capable of wirelessly transmitting data; and
iii) one or more remote computer servers in a cloud computing environment;
b) collecting a sample from at least one test subject, extracting a nucleic acid from the sample using a sample preparation device, and placing the extracted nucleic acid within the cartridge component of the isothermal amplification device or placing the sample within the cartridge component of the isothermal amplification device;
c) determining a presence of at least one prior or consensus nucleic acid sequence in the extracted nucleic acid or in the sample using the isothermal nucleic acid amplification reactions within the cartridge component of the isothermal amplification device by maintaining the specific range of temperature between 25 deg. C. to 80 deg. C., for a period of time from about 5 minutes to about 120 minutes in an environment of ambient temperature ranging from 0 deg. to 50 deg. C.; and
d) processing the isothermal amplification information using the mobile device, and transmitting said isothermal amplification information to the one or more remote computer servers in the cloud computing environment.

31. The method of claim 30, wherein said isothermal amplification information comprises one or more of isothermal amplification data, protocols, primer sets, analysis methods, solutions, and services.

32. The method of claim 30, wherein the mobile device further comprises computer readable storage medium configured to instruct the computerized system to perform executable instructions for isothermal amplification of nucleic acid of at least one test subject, and analyze and transmit the isothermal amplification information to the cloud computing environment.

33. The method of claim 31 wherein the service includes one or more of diagnosis of human, animal or plant's diseases, a treatment, a disease prevention, a report for the identity of origin of a test subject, environment surveillance, appraisement of a test subject.

34. The method of claim 30, wherein the cloud computing environment authenticates a user at a point-of-collection location.

35. The method of claim 30, wherein the cloud computing environment provides the manifest of at least one test subject to the user.

36. The method of claim 30, wherein the cloud computing environment provides a list of experts in a specific application for a user to consult or receive a service from.

37. The method of claim 30, wherein the cloud computing environment provides or receives inventory information of said test kit, said inventory information is used to inform a user for future purchase of said test kit.

38. An isothermal amplification device comprising:
a) a substantially hollow thermal container with a closed bottom end and an open top end capable to be sealed;
b) a heat source positioned within said thermal container, said heat source is able to maintain a specific range of temperature suitable for an isothermal amplification reaction; and
c) a test kit comprising a cartridge component positioned next to said heat source, and said cartridge component is suitable for conducting said isothermal amplification reaction within to generate an isothermal amplification information;
wherein said heat source or at least one component of said test kit is placed adjacent to a temperature indicating label, whereby the temperature of said heat source or at least one component of said test kit is monitored by a color change of said temperature indicating label.

39. The isothermal amplification device of claim 38, wherein said test kit further comprises a negative control sample or a positive control sample.

40. The isothermal amplification device of claim 38, wherein said heat source further comprises a phase change material which serves as a thermal storage medium.
